SEQUENCE LISTING (5 sequences)

SEQ ID NO: 1 - protein, 221 aa, Zingiber officinale
DDLPDSIDWRENGAVVPVKNQGGCGSCWAFSTVAAVEGINQIVTGDLISLSEQQLVDCTTANHGCRGGWMNPAFQFIVNNGGINSEETYPYRGQDGICNSTVNAPVVSIDSYENVPSHNEQSLQKAVANQPVSVTMDAAGRDFQLYRSGIFTGSCNISANHALTVVGYGTENDKDFWIVKNSWGKNWGESGYIRAERNIENPDGKCGITRFASYPVKKGTN

SEQ ID NO: 2 - protein, 221 aa, Zingiber officinale
  MISC_FEATURE (219)..(219): N = any amino acid
  MISC_FEATURE (220)..(220): N = any amino acid
  MISC_FEATURE (221)..(221): N = any amino acid
DVLPDSIDWREKGAVVPVKNQGGCGSCWAFDAIAAVEGINQIVTGDLISLSEQQLVDCSTRNHGCEGGWPYRAFQYIINNGGINSEEHYPYTGTNGTCDTKENAHVVSIDSYRNVPSNDEKSLQKAVANQPVSVTMDAAGRDFQLYRNGIFTGSCNISANHYRTVGGRETENDKDYWTVKNSWGKNWGESGYIRVERNIAESSGKCGIAISPSYPIKEXXX

SEQ ID NO: 3 - protein, 264 aa, bovine
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 4 - protein, 6 aa, chicken
PHNPGY

SEQ ID NO: 5 - protein, 8 aa, bovine
WGQPHGGG

(19) United States
(12) Patent Application Publication
(10) Pub. No.: US 2007/0264311 A1
(43) Pub. Date: Nov. 15, 2007

(54) Cysteine Protease from Ginger (Zingiber) as a Food Improver and Anti-Inflammatory

(75) Inventor: Clifford J. Hawkins, Kangaroo Point (AU)

Correspondence Address: THE MCCALLUM LAW FIRM, P. C., 685 BRIGGS STREET, PO BOX 929, ERIE, CO, 80516, US

(73) Assignee: NATBIO PTY LTD, KANGAROO POINT, QUEENSLAND AUSTRALIA (AU)

(21) Appl. No.: 10/590,530

(22) PCT Filed: Feb. 23, 2005
(86) PCT No.: PCT/AU05/00238

§ 371 Date: May 16, 2007

(30) Foreign Application Priority Data
Feb. 23, 2005 (AU) 2004901086
Feb. 24, 2004 (AU) 2004900929

(51) Int. Cl. A61K 36/906; A21D 10/00; A21D 13/00; A23K 1/10; C12C 11/00; C12N 5/08; C12N 5/06; A61K 47/00; A23L 1/03; A23L 1/223

(52) U.S. Cl. 424/439; 424/756; 426/11; 426/442; 426/549; 426/638; 426/652; 435/375

(57) ABSTRACT

The present invention relates generally to plant extracts and/or components isolated therefrom which exhibit desirable properties in relation to therapy and/or food technology. More particularly, the present invention relates to extracts and components isolated thereof from the plant genus Zingiber and in particular from the rhizome of the species Zingiber officinale (also known as ginger) which comprise activities having broad applicability in the fields of research reagents, inter alia pharmaceutical and/or nutraceutical product development, manufacture of improved high-value food and feed products, production of alcohol from cereals and waste treatment.

### BACKGROUND OF THE INVENTION

1. Field of the Invention

The present invention relates generally to plant extracts and/or components isolated therefrom which exhibit desirable properties in relation to therapy and/or food technology. More particularly, the present invention relates to extracts and components isolated thereof from the plant genus Zingiber and in particular from the rhizome of the species Zingiber officinale (also known as ginger) which comprise activities having broad applicability in the fields of research reagents, inter alia pharmaceutical and/or nutraceutical product development, manufacture of improved high-value food and feed products, production of alcohol from cereals and waste treatment.

2. Description of the Prior Art

Bibliographic details of the publications referred to by author in this specification are collected at the end of the description.

Reference to any prior art in this specification is not, and should not be taken as, an acknowledgment or any form of suggestion that this prior art forms part of the common general knowledge in any country.

Extracts from the tissues of monocotyledonous and dicotyledonous plants have provided a vast number of compounds and mixtures of compounds useful in medicine—including both Western-style and traditional approaches, such as those used in Sharmanism and Chinese medicine; building construction; the automotive industry, and biotechnology.

Particular extracts from the tissues of monocotyledonous and dicotyledonous plants are extremely useful, for instance, in the areas of food processing and food technology. In such areas, extracts made from plant tissues provide a very diverse range of additives and treatments for food, including spices, coloring, preservatives and condiments to flavour food and compounds to treat food to increase palatability. Extracts from plant tissues also provide compounds that can be used to enhance the long-term storage capacity and shelf life of manufactured and processed food.

In addition, there is increasing interest in identifying agents which are efficacious in improving the quality of food preparations, or that may be used separately or added to foods to produce a so-called “functional food”. Functional foods possess an identifiable quality or qualities associated with maintaining health and/or preventing deterioration thereof. An improved quality of an existing food may be, for example, a “no-fat”, “salt-reduced” or “allergen-free” equivalent of an existing food item. These concepts apply equally to feed for the many animal industries in addition to food for human ingestion. Furthermore, plant extracts are of use in a range of industrial applications that require, for example, a protein recovery phase, as well as in areas such as waste management.

The rhizome of the ginger plant, Zingiber officinale, has been used as a spice in food preparation and as a non-specific “herbal remedy” for various disease conditions, sometimes in conjunction with honey. Neither the efficacy nor the underlying activity, however, has been delineated or quantified in a manner permitting reliable reproducible outcomes, sufficient for consistent treatment purposes. Furthermore, studies designed to assess such presumptions have had to contend with the over-riding difficulty of lack of consistent and reproducible trial data.

One disease condition of interest is Celiac disease (also referred to as Coeliac disease and Celiac Sprue). In this condition, there is a reduced ability to digest wheat and there is often a direct toxic effect of gluten on the lining of the intestine called the intestinal mucosa. Symptoms may include diarrhoea, failure to thrive, short stature, discoloured dental enamel, depression, premature degeneration of the nervous system, seizures, arthritis, nutritional deficiencies due to malabsorption and abdominal distension.

Surprisingly, the subject inventors have identified a number of useful and varied applications including the use in treatment of Celiac disease for the various ginger rhizome extracts and/or components thereof. In accordance with the present invention, the difficulties associated with variability and lack of consistency of various extracts and components of the ginger rhizome have been overcome. This has enabled the quantification and characterization of the ginger rhizome and extracts thereof and its components.

### SUMMARY OF THE INVENTION

Nucleotide and amino acid sequences are referred to by a sequence identifier number (SEQ ID NO:). The SEQ ID NOs: correspond numerically to the sequence identifiers <400>1 (SEQ ID NO: 1), <400>2 (SEQ ID NO:2), etc. A summary of the sequence identifiers is provided in Table 1. A sequence listing is provided at the end of the specification.

The present invention provides extracts, and components thereof, derived from members of the Zingiberaceae plant family. Members of this family include, for example, Zingiber mioga, Zingiber officinale, Zingiber cassumunar and Zingiber zerumbet. Reference to “Zingiber extracts” in the subject specification includes extracts from all of the aforementioned species. The preferred species is Zingiber officinale, also known as ginger. The extracts and components, derived from the rhizome of the Z. officinale plant, comprise activities which are able to be applied usefully in a very wide range of related fields, extending from a processing aid and as an additive for animal and human feed/food and health maintenance, to disease prophylaxis and treatment. One particular example of a disease state is infection by a virus, bacteria or eukaryotic organism (e.g. fungus, yeast, lower eukaryote). Even more diverse areas, such as laboratory applications in the life sciences, including cell and molecular biology applications, and industrial applications such as the production of alcohol (e.g. ethanol) from cereals and the treatment of waste products, are contemplated, assessed and found to benefit from the application of the extracts and components of the present invention.

The present invention also provides Zingiber extracts such as ginger crush and dry ginger and Zingiber components such as Zingibain. Specific applications of the plant extracts and components of the present invention encompass, inter alia, food and feed processing; allergen inactivation such as the neutralisation of the cause of certain food intolerances; blood clot prevention and/or disintegration; wound healing, and prophylaxis and/or treatment in a range of disease conditions extending to cancer, inflammatory conditions including allergic and intolerant reactions, and the inhibition of virus infection.

In a particularly surprising embodiment, the extract is useful in the treatment and/or prophylaxis of Celiac disease (also known as Coeliac disease and Celiac sprue) and other forms of gluten intolerance.

The useful activities are found in one or more fractions derived from finely cutting and extracting (“crushing”) the ginger (Zingiber) rhizome. The resulting crush may be dried to generate an active powder form or, alternatively, may be filtered to produce a crush filtrate from which may be generated an “isolate” comprising the components referred to herein as “Zingibain”. In any of the foregoing cases—the crush, the dried powder, the crush filtrate or the isolate—the preferred active component comprised therein is Zingibain. Zingibain activity may be used consistently and reliably to hydrolyze, in a highly specific manner, a particular target. More particularly, Zingibain is effective in any situation wherein the target comprises a proteinaceous molecule that comprises a significant percentage of proline residues. The proline residues are preferably preceded and/or followed by a hydrophilic amino acid. Suitable amino acids include, for example, glutamine, arginine, lysine, asparagine, glutamic acid and aspartic acid. Reference herein to “Zingibain activity” may also be read as “Zingibain activities”.

As stated above, the preferred active component comprised within the extract or molecular components thereof is referred to herein as “Zingibain” and it has application in a wide range of related fields.

One field of application wherein the Zingibain activity of the present invention finds use is in industries engaged in the preparation of food and feed for humans and animals, respectively. Such foods include breakfast cereals, snack foods and functional foods. Zingibain may be used to improve the quality characteristics of edible material. Other fields of application extend to and include a means for the maintenance of health of animals, including human animals as well as companion and farm animals, and to prophylaxis and treatment of diseases/disorders of animals including humans. Additional applications encompass use of the molecular components as tools in cell and molecular biology, and industrial applications such as the production of alcohol (e.g. ethanol) from cereals and the treatment of waste products.

Particularly preferred edible materials are those which constitute feed and/or food for animals, including human animals. Resulting products are characterized in that they are, for example, more tender, more palatable, less allergenic or less likely to cause an intolerant reaction than their untreated equivalents. Food and feed products or ingredients thereof may have the improved quality characteristic conferred upon them by prior treatment with the extract and/or molecular components of the present invention.

In one embodiment, the tenderness of meat products, and the enhanced juiciness and greater density from an increased water binding capacity particularly in manufactured meats and small goods for human consumption is increased by prior treatment with the extract and/or molecular components of the present invention. Collagen, the major proline-containing protein of meat, is thereby degraded, leading to a consistency having greater tenderness. Further applications of the extracts and molecular components of the present invention relate to their use in degrading collagenous fibres in tissues wherein their presence is undesirable as, for example, in a cosmetic method of treatment designed to remove or reduce the presence of collagen in a target tissue.

The plant extract of the instant invention is further useful as a medicament or in the manufacture of a medicament for the treatment and, in some cases, prophylaxis of a disease condition of skin such as, for example, burns, insect bites and stings, abrasions, cancer, psoriasis and other inflammatory disorders and infection by viruses, bacteria, fungi, yeast or lower eukaryotes.

Skin disorders of the foregoing type typically involve superficial lesions and/or abnormalities that require topical application of a medicament useful in the treatment thereof. The instant invention, however, provides agents that may be formulated as medicaments for systemic administration, for example, as a powder, liquid, syrup, tablet, and capsule. Hence, the extract and/or molecular components thereof are also applicable for treatment and, in some cases, prophylaxis of a broader range of ailments extending to atherosclerosis, tumors, inflammatory diseases, including food intolerances such as gluten intolerance, particularly Celiac disease, prion-caused disease, forms of dementia, blood disorders, and the like and infection by pathogenic organisms and viruses.

In related embodiments, the extract and/or molecular components of the present invention may be applied in a method for the prevention and/or treatment of a range of disease states, including a systemic and/or skin disorder such as those recited above.

The extract and components of the present invention exhibit proteolytic activity directed, in particular, at targets adjacent to a conformationally exposed proline residue preceded and/or followed by a hydrophilic amino acid residue. Thus, Zingibain may be used consistently and reliably to hydrolyze such a target. Therefore, in addition to the applications described above, this property makes Zingibain especially useful in circumstances requiring consistent analytical-grade tools such as in research and development laboratories applying, for example, cell and molecular biological approaches to the investigation of biological questions. Such investigative approaches may require, inter alia, reproducible and complete removal of cellular material away from tissue culture containers; dissociation of tissue into single cells for harvesting; reliable target-specific protein degradation, and the like.

Furthermore, reliable target-specific protein degradation is one particular property of Zingibain, which is also sought after in industrial applications, such as in the hydrolysis of the gluten and related proteins in cereals to improve the efficiency of the hydrolysis and fermentation of starch to form ethanol, and in the treatment of waste products comprising unwanted proteinaceous material from plant and/or animal sources, wherein the complete dissociation of the waste material is desirable.

Another useful property of Zingibain is in its action on gluten and gluten-related proteins contained in bakery products and foods made from cereals. It is proposed that Zingibain acts on proteins and/or peptides to cleave them such that the peptides which are allergenic response triggers to persons intolerant to gluten are rendered digestible to such persons without adverse reactions.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

TABLE 1
Summary of sequence identifiers

| Sequence | |
|---|---|
| ID NO: | Description |
| 1 | Amino acid sequence of the component, isolatable from |
| | the ginger rhizome fraction designated GP-II, and |
| | exhibiting cysteine protease activity |
| 2 | Amino acid sequence of the dominant component, isolatable |
| | from the ginger rhizome fraction designated GP-I, and |
| | exhibiting cysteine protease activity |
| 3 | amino acid sequence of the bovine 28,600 Da infectious |
| | protein (prion) |
| 4 | Amino acid sequence of prion repeat unit from chicken |
| 5 | Amino acid sequence of prion repeat unit from bovine |

### BRIEF DESCRIPTION OF THE FIGURES

FIG. 1 is a representation of the structure of Zingibain, showing the four molecules in the crystallographic unit cell in two different orientations with the helical domains represented by cylindrical tube-like shapes and the β-sheet domains represented by flat rectangular shapes. The locations of the saccharide moieties are also indicated (Choi et al., Biochem. 38:11624-11633, 1999).

FIG. 2 is a graphical representation of the Zingibain activity for the hydrolysis of collagen as azocollagen as a function of temperature. Activity of Zingibain is expressed as A₅₂₀ units released per unit time on the y axis. The temperature is indicated on the x axis.

FIG. 3 is a graphical representation showing Meat Standards Australia Trials. A trial of 180 people from three community groups assessed meat quality from 20, 100% Brahman cattle from North Queensland with matching muscles treated with Zingibain and untreated (control). “Tend” is a tenderness score averaged over the cattle and the individual people scores given from 0 to 100. “Juice” is a juiciness score; “Oall” is an overall palatability score, each averaged in the same way for as for tenderness. MQ4 is a weighted score over the 4 parameters used by the MSA to give an overall grade to the meat.

FIG. 4 is a pictorial representation showing the trinodular structure of the fibrinogen molecule. (Refer to Retzinger, 2000; http://oz.uc.edu/˜retzings/fibrin( ).htm). It is a dimeric plasma protein, with each monomer unit being composed of disulfide linked chains Aα, Bβ and γ. The amino terminal of 16 residues of Aα and 14 residues of Bβ are called fibrinopeptides A (FpA) and B (FpB), respectively. The dimer is a 450 angstrom long “rope” with the amino-terminal chains forming a globular domain (the so-called disulfide or E knot) where 11 disulfide bonds hold the six chains together, and the carboxy-terminal chains for Bβ and γ end in the globular D domains, whereas the carboxy-terminal chains for Aα extend back into the central E domain. Except for the α-C domains, the regions between the globular domains form α-helical coiled-coil structures.

FIGS. 5A-C are graphical representations showing the change in weight over time of animals (dogs) when provided with food supplemented with Zingibain. (A) a 2.5-year-old Airedale Terrier bitch whose weight changed from 18.3 kg on Day 0 to 23.3 kg by Day 87; (B) a 5-year-old Kerry Blue Terrier dog; by Day 120, his weight had increased by about 4 kg from his minimum; (C) the fluctuation in weight of 7-year-old Miniature Schnauzer bitch correlated with the addition and removal of Zingibain supplement from her food. Refer to Example 7 for additional data and information.

### DETAILED DESCRIPTION OF PREFERRED EMBODIMENTS

The present invention is predicated in part on the observation that members of the Zingiberaceae plant family comprise an extractable fraction, which fraction or components thereof exhibit properties useful in a range of applications. The preferred species is Zingiber officinale, also known as ginger. Other members of the Zingiberaceae plant family are, however, not precluded and are intended to fall within the scope of the present invention. Examples of other species of Zingiber include Z. mioga, Z. cassumunar and Z. zerumbet. Reference hereinafter to a “ginger plant” is not intended to exclude species of Zingiber other than Z. officinale.

Preferably, the species of Zingiber is Z. officianale.

The present invention advantageously allows commercial quantities of the active ingredients from the extract and various components of the ginger rhizome to be prepared. This allows the active elements to be employed at concentrations not previously possible and/or without imparting the taste of ginger into the final product. The last point is particularly relevant given the strong and distinctive taste of ginger, which is not desirable in some contexts.

Furthermore, it renders possible for the first time cereal containing, more particularly gluten containing, food products for Celiac patients, wherein the food has a taste substantially similar or better than the corresponding food which has not been subject to treatment with the extract of the invention. Prior to the present invention the primary treatment of gluten intolerance and particularly Celiac disease was simply to avoid eating gluten-containing food. Furthermore, gluten free foods are limited in choice.

Thus, the present invention allows the preparation of traditional gluten containing foods, which are suitable for consumption by persons who are intolerant to gluten, for example those with Celiac disease.

The subject invention further permits the generation and/or processing of functional foods and a range of animal and human foods including snack foods.

The present invention identifies and delineates a wide range of useful applications for definable extracts and components from Zingiber such as Z. officinale. The extracts and components thereof are found to comprise inter alia hydrolytic activity, capable of acting highly specifically on proteinaceous molecules that comprise a conformationally exposed proline residue preceded and/or followed by a hydrophilic amino acid residue.

Reference to “Z. officinale” or “Zingiber officinale” or “ginger plant” is to be read as including other species or genera of the Zingiberaceae family which have similar properties.

The “molecular components”, which are comprised in and isolatable from an extract of the Zingiber, such as Z. officinale, rhizome, are enzymes the function of which is to degrade proteins. These enzymes are generally referred to as proteolytic enzymes or proteases, and they function by hydrolyzing peptide bonds within the amino acid sequences that constitute proteins. As will be well known to one skilled in the art, proteases are ubiquitous in nature and are many and varied in their structure and particular preferred substrate. They are, therefore, generally grouped into like kinds, according to their usual target.

One group of proteases is referred to in the art as “cysteine proteases”, in which a thiol group of a cysteine residue is the nucleophilic group involved in attacking and hydrolyzing a peptide bond. Representative members of the “cysteine protease” group of proteolytic enzymes include, for example, papain, bromelain and ananain, ficin and actinidin. These molecules are isolatable from papaya (Carica papaya), pineapple (Ananas comosus), figs, and kiwi-fruit (Actinidin chinensis), respectively. Zingibain is from the group of enzymes known as “cysteine protease”. More particularly, Zingibain is a proline-specific cysteine protease. Accordingly, Zingibain is effective in any situation wherein the target is a proteinaceous molecule that comprises a significant percentage of proline residues.

In the context of the present invention, “significant percentage” is to be understood as an amount of proline in excess of about 5%, which is higher than normal in proteins and which gives a greater chance of proline being preceded or followed by a hydrophilic amino acid residue in an exposed site for successful hydrolysis.

Preferably, the percentage of proline is less than about 60%, more preferably less than about 50%, even more preferably less than about 40%, still more preferably less than about 30%, and most preferably less than about 20%. Is it necessary to put in the last phrase because collagen has over 30% proline or hydroxyproline??) Hence, a reference herein to a “proline-containing protein” is to be understood to be a reference to a proteinaceous molecule that comprises a significant percentage of proline residues, as hereinbefore defined.

Accordingly, one aspect of the present invention is directed to the use of a rhizome from a species of Zingiber, preferably Z. officinale rhizome, in the manufacture of an extract or a molecular component thereof, which is capable of hydrolyzing proline-containing proteins including protein fragments (peptides).

Another aspect of the present invention, therefore, is directed to the use of Zingiber, such as Z. officinale rhizome in the manufacture of an extract or a molecular component thereof, which is capable of hydrolyzing proline-containing proteins and fragments thereof, for producing edible materials exhibiting improved quality characteristics.

The molecular components that provide the useful activity of the present invention are found in a fraction derived from finely cutting or otherwise comminuting rhizome of Zingiber such as Z. officinale.

A number of different formulations may be derived from processing the finely cut ginger rhizome. The cut tissue may be dried to generate the spicy ginger known to culinary aficionados. Alternatively, the finely cut rhizome may be extracted to produce a “ginger crush”, the solution of which comprises the desired active molecular components of the present invention.

This ginger crush may be dried to generate an active powder form or, alternatively, may be filtered to produce a crush filtrate from which may be isolated Zingibain, which is regarded herein as one of the molecular components of the ginger plant extract. In any of the foregoing formulations—the dried powder, the crush or its filtrate or the isolate—the preferred activity is due to a Zingibain extract. Reference herein to “molecular components” includes a component or extract having the characteristics of Zingibain.

“Zingibain”, as used herein, refers to a protein fraction, isolatable from ginger rhizome, and comprising proteolytic activities of at least one or two or three closely related enzyme fractions, separable by, for example, DEAE-cellulose chromatography. One of the fractions comprises the GP-II proteases. Another fraction, referred to as “GP-I”, comprises two highly homologous proteases. All three proteolytic enzymes are comprised in the dried powder, the crush or its filtrate or the isolate as described herein. Hence, reference to “molecular components” is a reference to any one of or, alternatively, all three proteolytic enzymes. Similarly, throughout this specification, a reference to “Zingibain” is to be understood to be a reference to the unseparated protease fraction comprising all three protease enzyme activities, or to any one or more of the said protease activities.

Without intending to limit the present invention to any one theory or mode of action, it is proposed that Zingibain degrades its protein targets by hydrolyzing peptide bonds between an amino acid residue following a proline and the next amino acid residue thereafter in the amino acid sequence, reading from the N-terminal.

For optimal hydrolytic effect, a proline residue is preferably preceded and/or followed by a hydrophilic amino acid. Suitable hydrophilic amino acid residues include, for example, glutamine, arginine, lysine, asparagine, glutamic acid and aspartic acid.

The term “hydrolyzing” as applied to the effect of a proteolytic enzyme on a peptide bond means that the affected peptide bone is broken or destroyed, and the sequence of the hydrolyzed protein is thereby severed or cleaved at that point in the chain. An attacked protein may be broken down, through hydrolysis, into two or into many peptide pieces, depending on the extent of suitable bonds for hydrolysis and on the extent of hydrolysis that actually occurs. Hydrolysis, therefore, destroys proteinaceous material and results in its conversion and/or degradation into smaller cleaved portions of protein, or peptides, and/or, in its most extreme form, into the amino acid constituents thereof. Destroyed, degraded, converted, cleaved, and/or hydrolyzed proteinaceous material no longer exhibits its naturally occurring function.

Zingibain's specificity for hydrolyzing proteins adjacent to proline results in the splitting of the protein rather than in the break-down of proteins to small peptide pieces or individual amino acids. However, if the substrate for the Zingibain is a peptide, for example, which is the product of digestion/hydrolysis of other proteolytic enzymes such as those found in the stomach including enzymes like trypsin and/or chymotrypsin, then the product after hydrolysis with Zingibain may be individual amino acids and/or di- or tri-peptides.

In accordance with the present invention, ginger rhizomes may be processed to generate extracts that comprise the proline-specific cysteine protease, Zingibain, which is capable of destroying and/or degrading proteins via hydrolysis adjacent to or “following” proline residues.

Accordingly, another aspect of the present invention contemplates the use of Zingiber, such as Z. officinale, rhizome in the manufacture of an extract, wherein said extract comprises the proline-specific cysteine protease, Zingibain, for producing edible materials exhibiting improved quality characteristics.

In a related embodiment, the present invention contemplates the use of an extract of the Zingiber such as Z. officinale rhizome, wherein said extract comprises molecular components such as the proline-specific cysteine protease, Zingibain, in the manufacture of edible materials exhibiting improved quality characteristics.

The extract may be added in the food, for example, a bakery product such bread in the range 0.01 to 10% by weight. Preferably it is added in the range about 0.03 to 5%.

As used herein, the term “extract” extends to and encompasses any formulation, derived from the Zingiber, such as Z. officinale, rhizome, in which Zingibain exists and may be used in accordance with the present invention. “Extract” therefore extends to dried, powder, ginger crush, crush filtrate and isolate, as described above, and any other suitable formulation. The terms “extract” and “Zingibain” are used herein interchangeable.

The uses according to the present invention includes use of a protein substantially identical to Zingibain regardless of its source, for example, regardless of whether it is prepared recombinantly, synthetically and/or probiotically in situ. Substantially identical in the context of this specification will be understood to mean at least 95% identity with the sequences No. 1 or No. 2 at the amino acid level. Preferably substantially identical will be 98% identity and more preferably 99% identity.

In accordance with the methods and applications of the present invention, ginger extracts may be applied usefully in the manufacture of edible materials that exhibit improved quality characteristics. “Edible materials” includes and encompasses animal- and/or plant-derived matter, which is used in the preparation of any item to be consumed by animals, including human animals, as a nutrient source. Edible materials includes the ingredients that are used in the preparation of manufactured feed and/or food items and extends to and encompasses the manufactured feed and/or food items so prepared. The term “feed” generally refers to such items when consumed by animals other than humans; correspondingly, the term “food” generally refers to such items when consumed by humans. In this context, “animals” includes both companion animals, such as cats, dogs and horses, and production animals, such as pigs, goats, sheep, chickens, aquatic species and cattle, inter alia.

Common edible items are comprised, for example, of grains such as cereals and legumes, and meat as well as meat-derived manufactured products. Any other feed and/or food item, which is consumed as a source of nutrients, falls within the scope of this aspect of the present invention.

Accordingly, the present invention provides for the hydrolysis of proline-rich proteins comprised within edible material, the result of which is the improvement in the processing of the material and in one or more quality characteristic thereof.

“Quality characteristics” generally relate both to more readily quantifiable characteristics such as nutritive value and digestive value, and to more qualitative characteristics such as taste value. Examples of quantifiable characteristics related to nutritive value and/or digestive value include, inter alia, total fat content, extent of fat distribution, presence of allergen-causing ingredients or food intolerant epitopes, prion content and shelf-life etc. Examples of qualitative characteristics related to taste value include, inter alia, juiciness, tenderness, texture and colour.

Bakery products such as bread may show improved texture, smoothness, increased water content of up to 4% (which can be desirable), increased uniformity and improved crust when the extract is employed. Therefore, in one aspect the subject invention provides an improver for a bakery product comprising a ginger extract as described herein.

In a further aspect the invention provides a method for preparing a bakery product such as a bread product comprising the steps of:
a) mixing an improver comprising an extract of Zingiber including at least one cysteine protease, according to the invention, with further ingredients of the bakery product and forming a dough or mixture or batter;
b) if required, allowing the dough or mixture or batter to rest; and
c) comminuting the dough if required, shaping and baking the dough or mixture or batter to form the bakery product.

The process may include the additional step of preparing an improver comprising an extract of Zingiber including at least one cysteine protease and optionally including one or more enzymes employed in food preparation, for example, enzymes selected from the group consisting of xylanase, amyloglucosidage, lipase and maltogenic amylase.

The subject invention particularly provides an improver for a bakery product comprising:
i) an extract of Zingiber including at least one cysteine protease
ii) 0-10% xylanase;
iii) 0-10% amyloglucosidage;
iv) 0-10% lipase; and
v) 0-10% maltogenic amylase such as Novamyl®
   wherein the percentages above are expressed as % w/w ratios relative to the total weight of the improver formulation and with the proviso that ii) to v) do not all represent 0% simultaneously.

The improving agent is prepared by combining the extract with the other optional ingredients, which may then be subjected to a conventional mixing process. The improver once formulated can be incorporated into the ingredients employed in, for example, the bread product prior to forming the dough. The components of the improver may be varied according to the intended use. The actual selection of ingredients and quantities is dependant on the properties which the improver is intending to impart on the style of bakery product. For long fermentation time to no fermentation time doughs, improvers differ in levels of enzymes to control extent of dough expansion. Improvers for weak flours differ to improvers for strong flours in that different types and/or levels of emulsifiers and enzymes are used to achieve the same bread quality. For example, baguettes and white pan bread may require 0.5 to 1.5% w/w, whole meal bread may require 2 to 5% w/w, multi-cereal bread may require 3 to 7% w/w and panettone may require 2 to 4% w/w relative to the amount of flour employed.

In one embodiment of the invention a 3 Kg commercial batch of bread, for example, may contain between 1 and 120 mL of ginger crush.

A further advantage of the improver according to the invention is that the mixing time of the dough, mixture or batter may be decreased in comparison to formulation which does not contain the extract.

Further characteristics that may be improved through the application of the extract and/or molecular components of the present invention include fat distribution and content; allergenicity; food intolerance, prion content; and feed conversion.

Allergenicity and food intolerance are generally related to the presence of particular proteins in, for example, grains such as cereals (wheat, oats, barley, rye, sorghum, corn inter alia), legumes (chick pea, soybean, lentil, peanuts, inter alia), and dairy products such as proline-rich proteins that, upon ingestion, elicit an allergic or intolerant antigenic response. By way of example, grains comprise a proline-rich storage protein, localised in the endosperm, known as glutelin.

Wheat, for example, comprises a proline-rich glutelin, called glutenin. Glutelins are storage proteins located in the endosperm. They are rich in asparagine, glutamine, arginine and proline, and are low in lysine, tryptophan and methionine (Abrol et al., Aust. J. Agric. Res. 22: 197-202, 1971; Derbyshire et al., D. Phytochemistry 15: 3-24; 1976; supra; Kirkman et al., J. Sci. Food Agric. 33: 115-127; 1982; Larkins, B. A. “Seed storage proteins: characterization and biosynthesis” in “The Biochemistry of Plants” Stumpf, P. K.; Conn, E. E. (eds) Academic Press NY, Vol 6, pp 449-489). Wheat's glutenin is a large polymer with a molecular weight greater than a million. When the disulfide bonds are reduced, two fragments are isolated, a high molecular weight sub-unit with a molecular weight of 80-160 kDa, and a low molecular weight sub-unit similar to α-gliadin. Gliadin is another endosperm storage protein belonging to the group of molecules, unique to seeds of cereals and other grasses, known as prolamins that together with the glutenins are known to cause ‘gluten’ intolerance and associated auto-immune diseases in animals including humans with genetic predisposition to the intolerant reaction. Wheat α-gliadin has five domains, the first of which comprises a non-repeat N-terminus sequence plus a repeat sequence rich in glutamine, proline and aromatic amino acids.

All epitopes associated with ‘gluten’ intolerance that have been identified for glutenin and the gliadins have at least one proline in the epitope sequence adjacent to a hydrophilic amino acid residue (Vader et al., Gastroenterology 122: 1729-1737; 2002). Hydrolysis with Zingiber extracts such as Zingibain degrades these sequences, concomitantly removing the ‘gluten’ intolerance effect of the intact protein. Flour, such as wheat flour, can thereby be made ‘gluten-safe’ employing the extract of the present invention. Flour in the context of this application includes waxy flour which has a low amylose content.

These proteins are candidates for hydrolysis by the extracts of the present invention, being rich in proline sites suitable for attack by Zingiber extracts such as Zingibain. Hydrolysis of these proteins by Zingiber extracts such as Zingibain has been found to avert allergic and intolerant responses in patients with an existing sensitivity to gluten. By the addition of the ginger extract or isolate during processing to products such as baked goods, including: breads, which includes all types of leavened dough breads, in particular: square, high top, long ferment, bread rolls, baguettes, hamburger buns, grain breads, flat breads and so called no time doughs, which do not required fermentation before processing; cakes; muffins and English muffins; crumpets; pizza bases; buns and sponge and dough used in the preparation of the same, inter alia for human consumption or pelletised animal feed inter alia for animal consumption, an otherwise present or expected allergic or intolerant response in an affected person or animal may be avoided (refer to Examples 4, 5 and 6).

Thus, in a further aspect the invention provides a food product, for example, a gluten containing food product such as breads, cakes, pasta, pizza bases, noodles, breakfast cereals and the like comprising the said enzyme/extract or a composition containing the same according to the present invention.

Thus in a preferred aspect the invention a gluten containing food product such as bakery product wherein the gluten is cleaved through the portions of the gluten sequence that generates the peptide fragments which are toxic to gluten intolerant persons, such as those with Celiac disease i.e., wherein the gluten is degraded to render it non-toxic.

Preferably the gluten containing product is a bread product. Bread is a product which is obtained by baking yeast leavened dough prepared with flour and water with or without salt, edible fats, milk and other permitted food additives.

The ability of Zingibain to cleave known epitopes for allergic or intolerant effects in food, without destroying the palatability of the food is not restricted to foods made from cereal grains. There are similar epitopes in proteins in milk, soy, and peanuts, for example, that are known to cause allergies or intolerant reactions.

The active enzymes/extract may be applied to the final food product, for example, as a powder, liquid in a suitable formulation before consumption, in which situation the enzyme may be consumed in its “active” form.

Alternatively the enzyme/extract can be added to the component materials, such as flour, of the food product during preparation. The enzyme/active extract may then be deactivated during the cooking/final preparation of the food product. Cooking temperatures over 65° C. are thought to deactivate the enzymes.

This is advantageous in that the final food product does not need to be stored under special conditions to ensure the activity of the enzyme is maintained.

It also gives the food manufacturer control to ensure a food of consistent quality is manufactured and also avoids the end user being in control of the amount of enzyme that is dispensed and/or ingested.

Furthermore, this aspect of the invention has the additional advantage that it is unlikely that regulatory approval for such a food product would need to be obtained.

Thus the enzyme/extract may be in the active or deactivated form when the food containing it is consumed. Preferably the enzyme/extract will be in the deactivated form.

In a further aspect the invention provides the addition of the ginger extract such as Zingibain to a bulk material such as flour during processing. If necessary the enzyme could be deactivated after application to the material, which can then be handled, distributed and processed in the usual way. Thus, the invention extends to a bulk material treated with the enzyme/extract or composition containing same according to the invention and processes involving preparation of the same.

Still other useful properties of the subject extract is its ability to reduce fat content to from about 1% to 10% by weight including from about 1% to 5% by weight such as 1, 2, 3, 4 or 5%. Water content is also elevated to from about 1% to about 10% vol/wt such as from about 1% to 5% vol/wt (e.g. 1, 2, 3, 4, or 5%).

The subject invention also extends to use of such a bulk product in the preparation of a food product with a reduced propensity to cause an allergic and/or intolerant reaction in the relevant percentage of the population.

The bulk product, for example may be sold in, sacks or a loose material as may animal feed treated with the extract of the invention.

The enzyme may be allowed to act on the material/food for a period sufficient to effect the desired hydrolysis such as about 1 minute to about 24 hours, such as about 5 minutes to about 2 hours. Preferably the enzyme will be allowed to act for a period in the range about 5 to about 30 minutes.

In a further aspect the enzyme or extract may be administered as a pharmaceutical formulation or a health supplement type formulation to be taken, before, concomitant with or after the consumption of food.

Preferably an oral formulation is administered between 1 and 20 minutes before the consumption of food, such as 5 to 10 minutes before the consumption of food.

A health supplement formulation, for example, may be taken once or twice daily such as in the morning.

Health supplements according to the present invention are advantageous in that they aid general digestion of proteins and absorption of nutrients from food. This aspect of the invention may have particular application in poorer countries around the world, which have difficulty in providing adequate nutrition for their inhabitants.

Whilst not wishing to be bound by theory it is thought that, the ginger extract such as Zingibain when administered orally, for example, as a suitable formulation, can retain its activity and hydrolyze the toxic peptides in the gut thereby preventing them triggering an intolerant/allergic/inflammatory response. This in turn avoids the “autoimmune-type” damage inflicted on the body from such responses.

Thus the subject invention extends to compositions, preferably pharmaceutical and/or health supplement formulations comprising or consisting of said enzyme/extract. The enzyme extract may be formulated as a tablet, capsule, powder, drink or the like. However, the enzyme may need protecting to survive to the acidic conditions of the stomach, for example, by enteric coating or buffering.

Alternatively the enzyme may be made in situ in the gut by yeast or bacteria designated/engineered to synthesise the enzyme/components.

The yeast and/or bacteria may be administered in the form of an active drink. Thus the invention also extends to probiotic formulations capable of preparing the enzyme in vivo.

Probiotic formulations according to the invention may be a fermented product derived from milk or soy or similar. Such formulations may include lactose, glucose, stabilisers and one or more flavourings. Yeast and bacteria which may be employed in the probiotic formulations are known to persons skilled in the art. Preferably the bacteria are lactobacillus such as lactobacillus casei.

Compositions as referred to herein are characterised by the presence of one or more excipients such as a diluent or carrier.

The plant extract of the instant invention is therefore useful as a medicament or in the manufacture of a medicament for the treatment and, in some cases, prophylaxis of food intolerances such as gluten intolerance, particularly Celiac disease and/or ulcerative colitis and/or inflammatory bowel disease.

Furthermore, the extract according to the present invention may therefore be useful in the treatment of symptoms such as diarrhoea, which may be a symptom of these diseases. Zingibain's specificity and temperature-activity profile for protein hydrolysis allow the qualities of food to be improved in a very controlled way during the food's cooking process and can result in higher cooked weight, shorter cooking times, and lower energy expenditure.

One particular application in this field relates, therefore, to the processing and tenderizing of any material containing the proline-rich natural protein, collagen. Resulting products are characterized in that they are more tender or, alternatively, more palatable than their untreated equivalents. Products amenable to such processing and improvement are generally meat and/or meat-derived products. Palatability may include measures of, for example, juiciness of meat products. Tenderizing of meat may be achieved through the inclusion, in the animal feed or during the manufacturing process, of the application of the extract and/or components of the present invention. Alternatively, the said extract may be administered in a suitable form to the edible meat material just prior to ingestion and/or preparation for ingestion such as by cooking. One way in which the desired quality improvement may be achieved is through the addition of Zingibain to sauces, marinades and/or stocks, for example.

In this context, the terms “meat” and “meat-derived” also extend to and encompass the flesh tissue of seafood; in particular, that which comprises edible material.

Other proline-rich proteins, suitable for attack by Zingibain, are also found in plant pollens and, in particular, in plant pollens that are highly allergenic.

The presence of the recently identified highly infectious protein named “prion”, in food and/or feed products, has been a critical and hugely expensive problem in, especially, the beef cattle industry of Europe and, in particular, the United Kingdom. The spread of the prion-caused infectious disease known as bovine spongiform encephalitis (otherwise known as “mad cow disease”) throughout that entire country led to the forced destruction of a high percentage of the industry of that country and to significant economic hardship. Moreover, there was widespread concern about the possibility of its spreading into the human population, through ingestion of contaminated food products.

Importantly, in the present context, the structure of prion shares some features with collagen, including the presence of a repeat region that contains proline in an amino acid unit that is repeated. In chicken prion, for example, there is a 54-amino-acid region with nine repeat units (PHNPGY) in which proline is every third amino acid, thereby forming an extended polyproline II helix, as is also found in collagen. While normal prion protein is protease-sensitive, the infectious conformation resists breakdown with many proteolytic enzymes. Given its proline-rich structure, however, prion presents an ideal target for hydrolytic degradation by Zingibain, which preferentially and specifically destroys proline-rich natural proteins. Prior treatment of meat and meat-derived products, therefore, presents a potential means of rendering the meat “prion-free” and, hence, safe for consumption.

Fat content and distribution through muscle tissue, and feed conversion, may also be advantageously affected by similar applications of the extract and/or components of the present invention.

Edible material which displays values such as decreased allergenicity, with reduced risk of intolerant reactions and decreased fat content, and reduced or eliminated prion content, are regarded as providing a healthier alternative to an equivalent product which does not exhibit such characteristics. Such “improved quality characteristics” are therefore sought after and edible material, comprising one or more of these characteristics, is generally preferred by consumers, manufacturers and health educators alike. In some instances, feed/food comprising one or more of these characteristics may be regarded as “functional food”. As will be obvious from the above, these quality characteristics apply equally to feed for farm and companion animal consumption as to food for human ingestion.

As already mentioned, one particularly proline-rich natural protein is collagen. Collagen is the most abundant protein in humans, accounting for about 25% of all protein, and its structure is largely conserved in the animal kingdom from the most primitive animals to humans. It is expressed in fibroblast cells. It forms the organic mass of tissues such as skin, tendon, blood vessels, bone, the cornea and vitreous humor of the eye, and basement membranes. In certain circumstances, it may be desirable or critical to remove and/or reduce the amount or presence of collagen from a particular tissue site. Examples include collagen fibres entangled in blood clots and in dead tissue around burn wounds.

Accordingly, a related embodiment of the present invention is directed to the use of Z. officinale rhizome in the manufacture of a medicament comprising an extract or a molecular component thereof, which is capable of hydrolyzing proline-containing proteins, for the removal or reduction of collagen in a target tissue.

In an alternative embodiment, the present invention is directed to the use of an extract of the Z. officinale rhizome, wherein said extract comprises molecular components capable of hydrolyzing proline-containing proteins, in a cosmetic method of treatment designed to remove or reduce the presence of collagen in a target tissue.

“Target tissues” include any tissue wherein collagen is present and wherein, for cosmetic or health purposes, the extent or amount thereof should preferably be, to a greater or lesser extent, reduced. Target tissues contemplated herein include those already cited above; namely, skin, tendon, blood vessels, bone, the cornea and vitreous humor of the eye, and basement membranes. Other tissues, however, may also be encompassed within the intended scope of the present invention, provided that the removal and/or reduction of the amount or presence of collagen from a particular tissue site is desirable and/or critical, and that it may be achieved by the hydrolysis with Zingibain.

Collagen, especially in its solubilized form, is an ingredient in cometic and medical preparations. Zingibain has an application in refining the structure of collagen fibres to make them more soluble, and consequently of greater value for cosmetic and medical preparations.

Without limiting the invention to any one theory or mode of action, it is proposed that the extract or molecular component of Zingiber, such as Z. officinale, rhizome specifically hydrolyzes proteins that comprise a significant percentage of proline residues. Particularly preferred proline-rich natural proteins include, but are not limited to, collagen, glutelins, prolamins, casein, prion, fibrin, fibrinogen, amyloid beta protein precursor, and particular cell membrane proteins including receptors etc., inter alia. Since these molecules are involved in many cellular and biochemical processes, the ginger rhizome extract referred to herein as Zingibain has applications, even more widely, in preventing and/or treating the effects of biochemical processes that may be undesirable and/or deleterious to health. Such processes may be superficial—affecting, for example, skin—or they may be systemic.

The plant extract of the instant invention is therefore useful as a medicament or in the manufacture of a medicament for the treatment and, in some cases, prophylaxis of a disease condition of skin such as, for example, burns, insect bites and stings, abrasions, cancer, psoriasis and other inflammatory disorders.

Accordingly, another aspect of the present invention is directed to the use of an extract of the Z. officinale rhizome, wherein said extract comprises molecular components capable of hydrolyzing proline-containing proteins, in the preparation of a medicament for the prophylaxis and/or treatment of a skin disorder or other disorder described herein, in a subject.

Reference herein to “prophylaxis” and “treatment” is to be considered in its broadest context. The term “treatment” does not necessarily imply that a subject is treated until total recovery. Similarly, “prophylaxis” does not necessarily mean that the subject will not eventually contract a disease condition. Accordingly, prophylaxis and treatment includes amelioration of the symptoms of a particular disorder or condition, or preventing or otherwise reducing the risk of developing a particular disorder or condition. The term “prophylaxis” may be considered as reducing the severity or the onset of a particular disorder. “Treatment” may also reduce the severity of an existing condition.

In this context, a “subject” may be a human or an animal subject.

Skin disorders of the foregoing type, which may be amenable to prophylaxis and/or treatment in this manner, typically involve superficial lesions and/or abnormalities that require topical application of a medicament useful in the treatment thereof. Such disorders include, for example, burns, insect bites and stings, abrasions sun damage and the like. However, it should be understood that the present invention is not limited thereto but extends to encompass more serious diseases, such as cancer, psoriasis and other inflammatory disorders.

Accordingly, yet another aspect of the present invention is directed to a method of treating and/or preventing a skin disease and/or abnormality in a subject, said method comprising contacting said diseased and/or abnormal skin with an effective amount of a medicament comprising an extract of the Z. officinale rhizome, wherein said extract comprises molecular components capable of hydrolyzing proline-containing proteins, for a time and under conditions sufficient to prevent, ameliorate or otherwise reduce symptoms of said disease and/or abnormality.

In this regard, the instant invention provides agents that may be formulated as medicaments for systemic administration. Hence, the extract and/or molecular components thereof are also applicable for treatment and, in some cases, prophylaxis of a broader range of ailments extending to atherosclerosis, tumors, inflammatory diseases such as inflammatory bowel disease and ulcerative colitis, prion-caused disease, dementia, blood disorders, viral infection, food intolerances such as Celiac disease, Crohn's etc.

Accordingly, a further aspect of the present invention contemplates the use of Z. officinale rhizome in the manufacture of a medicament comprising an extract or a molecular component thereof, which is capable of hydrolyzing proline-containing proteins, for the prophylaxis and/or treatment of a systemic disorder in a subject.

One possible life-threatening event wherein the application of Zingibain may provide effective prophylaxis is the transmission of infectious prion proteins through, for example, blood transfusion and/or tissue transplantation and/or contaminated surgical equipment or blood processing equipment. As mentioned above, while the infectious prion protein conformation resists breakdown with many proteolytic enzymes, its proline-rich structure makes prion an ideal target for destruction by Zingibain. Without wishing to limit this aspect of the invention to any one theory or mode of action, it is proposed that infectious prions may be transmitted to blood and/or transplant recipients or patients undergoing surgery, where they cause disease by inducing normal prion molecules to change conformation to the disease-causing structure. The transmission of Spongiform Encephalopathies in this manner is a major concern as the prion would be very difficult to detect, and the disease takes many years to produce symptoms. Prior treatment of blood/tissue with Zingibain and decontamination of surgical and blood-processing equipment obviates this dangerous possibility.

Thus in one embodiment, the present invention contemplates a method of treating and/or preventing a systemic disorder, said method comprising administering to a subject in need thereof an effective amount of a medicament comprising an extract of the Z. officinale rhizome, wherein said extract comprises molecular components capable of hydrolyzing proline-containing proteins, for a time and under conditions sufficient to prevent, ameliorate or otherwise reduce symptoms of the disorder.

Other disorders, in addition to those mentioned above, wherein the application of the extract of the present invention may be efficacious include other blood disorders, atherosclerosis, food intolerance and consequent auto-immune diseases, cancer tumors, whether or not metastatic, dementia, inflammatory diseases and viral infections. All such disorders are to be understood as being encompassed by the term “systemic”.

By way of further example, other particularly preferred proline-rich natural proteins, susceptible to proteolytic degradation by the extracts of the present invention include, in addition to collagen and prion, fibrin and fibrinogen. Fibrin and fibrinogen have extremely important functions in animals and, at the same time, are associated with the occurrence of some of the more common diseases, such as thrombosis, inflammation, cancer, and atherosclerosis. For example, polymerised, cross-linked fibrin forms blood clots causing thrombosis.

These proteins—fibrin and fibrinogen—also have proline residues in key positions, immediately preceded and/or followed by suitable hydrophilic amino acid residues, and are therefore susceptible to effective hydrolysis by the ginger extracts such as Zingibain-comprising formulations contemplated herein. Therefore, life-threatening clots may be degraded. Furthermore, by hydrolyzing fibrinogen and other members of the blood-clotting cascade, such as prothrombin, Zingibain is also able to prevent the formation of blood clots.

Fibrin and fibrinogen are also closely associated with inflammation. As used herein the term “inflammation” should be interpreted in its broadest sense to indicate a protective response of the body to tissue injury or destruction. Again without wishing to limit the present invention to any one theory or mode of action, it is understood that thrombin and factor XIIIa, which are generated immediately at a site of tissue damage, convert intra- and extravascular fibrinogen at the site to cross-linked fibrin. The fibrin meshwork entraps blood cells, limiting blood loss from the site. It further confines to the site inflammatory cells such as, for example, platelets, granulocytes, monocytes and lymphocytes, which would otherwise circulate. Some of these cells express on their outer surface cellular adhesion molecules that, when activated, have significant affinity for fibrin and/or fibrinogen. It is therefore proposed that, within the inflammation site, fibrin and/or fibrinogen is able to adhere to a variety of cells, thereby keeping them in the location of the inflammation.

As a consequence of their role in inflammation, fibrin and/or fibrinogen are also important in the promotion of tumor growth. Moreover, they have also been implicated in atherosclerosis, another disease associated with inflammation. Without limiting the application of the invention to any one theory, the atherosclerosis plaque consists of a deposit of extracellular hydrophobic lipids, lipid-laden macrophages, smooth muscle cells and proteins embedded just beneath the endothelial lining of large arteries, including fibrinogen and its degradation products. There is a positive correlation between the fibrin and/or fibrinogen content of the plaque and its lipid content, and plasma fibrinogen level is an independent risk factor for atherosclerotic cardiovascular disease.

Hence, targeted proteolytic degradation of fibrin and/or fibrinogen by the extracts of the present invention may be used to reduce and/or eliminate an inflammatory response, in situations where its occurrence and/or extent is inappropriate, unwanted and/or undesirable. Undesirable effects of fibrin and/or fibrinogen—including blood clotting, inflammation, atherosclerosis and tumour growth—may be prevented, ameliorated or otherwise reduced by the application of the Zingiber, such as Z. officinale, rhizome extract and/or components thereof referred to herein as Zingibain.

Virus cell-membrane proteins commonly are proline-rich and have multiple sites for hydrolysis by Zingibain. These proteins are essential for host-cell invasion and other functions, and their cleavage by Zingibain inhibits the viral infection.

The active component of the medicament is contemplated to exhibit therapeutic activity when administered in an “effective amount” that depends on the particular case. By “effective amount” is meant an amount necessary to at least partly obtain the desired response, or to delay the onset or inhibit progression or halt altogether the onset or progression of a particular condition being treated. The amount varies depending upon the health and physical condition of the subject being treated, the taxonomic group of the subject being treated, the degree of protection desired, the formulation of the composition, the assessment of the medical situation and other relevant factors. It is expected that the amount will fall in a relatively broad range, which may be determined through routine trials. Considering a human subject, for example, from about 0.1 mg to about 4 mg of active component may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

In accordance with the applications of the present invention, medicaments comprising the extracts and or components disclosed herein may be formulated, for use in conjunction with the instant methods, via topical administration or via systemic administration, depending on the nature of the subject's disorder. Appropriately formulated medicaments may then be utilised in the treating and/or preventing disease, whether a skin abnormality or disease, or a systemic disorder such as those referred to above. Such medicaments may be administered to a subject in any one of a number of conventional dosage forms and by any one of a number of convenient means. As already mentioned, “subject” may refer to any animal including but not limited to a human.

Contemplated suitable dosage forms of the active component include tablets, troches, pills, capsules, creams, oils, gels and the like, all of which may also contain additional components, as follows: a binder such as gum, acacia, corn starch or gelatin; excipients such as dicalcium phosphate; a disintegrating agent such as corn starch, potato starch, alginic acid and the like; a lubricant such as magnesium stearate; a sweetening agent such as sucrose, lactose or saccharin; a flavouring agent such as peppermint, oil of wintergreen or cherry flavouring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the active ingredient, its use in the therapeutic composition is contemplated. Supplementary active ingredients can also be incorporated into the compositions.

The active component may be administered in a convenient manner such as by oral, intravenous (where water-soluble), intra-peritoneal, intramuscular, subcutaneous, intra-dermal or suppository routes, or via implanting (e.g. using slow release molecules).

Suitable amounts of active ingredient for oral dosage forms may include 1 to 500 mg per unit dose, such as 10 to 250 mg per unit dose such as 50, or 100 mg.

Alternatively, the active component may be formulated for administration topically, such as by cream, oil or gel. The active component may be administered in the form of pharmaceutically acceptable non-toxic salts, such as alkali or alkaline earth salts, such as sodium, potassium, magnesium or calcium. The active component may be administered as a supplement to prepared food or drink.

Preferred formulations for topical administration include those in which the active component of the present invention is in admixture with a topical delivery agent such as lipids, liposomes, fatty acids, fatty acid esters, steroids, chelating agents and surfactants. Preferred lipids and liposomes include neutral (e.g: dioleoylphosphatidyl DOPE ethanolamine, dimyristoylphosphatidyl choline DMPC, distearoylphosphatidyl choline), negative (dimyristoylphosphatidyl glycerol DMPG) and cationic (dioleoyltetramethylaminopropyl DOTAP and dioleoylphosphatidyl ethanolamine DOTMA).

For topical or other administration, the extract and/or components of the present invention may be encapsulated within liposomes or may form complexes thereto, in particular to cationic liposomes. Alternatively, the extract and/or component may be complexed to lipids, in particular to cationic lipids. Preferred fatty acids and esters, pharmaceutically acceptable salts thereof, and their uses are known, such as are described in U.S. Pat. No. 6,287,860.

The extract and components of the present invention exhibit proteolytic activity directed, in particular, at targets which comprise a significant percentage of proline residues. The proteolytic activity, referred to herein as Zingibain, may be used consistently and reliably to hydrolyze such a target. Therefore, in addition to the applications described above, this property makes Zingibain especially useful in circumstances requiring consistent analytical-grade tools such as in research and development laboratories applying, for example, cell and molecular biological approaches to the investigation of biological questions. Such investigative approaches may require, inter alia, reproducible and complete removal of cellular material away from tissue culture containers; dissociation of tissue into single cells for harvesting; or reliable target-specific protein degradation, etc.

Accordingly, still another aspect of the present invention contemplates the use of Z. officinale rhizome in the manufacture of an extract or a molecular component thereof, which is capable of hydrolyzing proline-containing proteins, for tissue dissociation and/or harvesting of dissociated cells.

In a related embodiment, the present invention contemplates the use of Z. officinale rhizome in the manufacture of an extract or a molecular component thereof, which is capable of hydrolyzing proline-containing proteins, for specific cleavage of an identified target.

Furthermore, reliable target-specific protein degradation is one particular property of Zingibain, which is also sought after in industrial applications, such as in the production of ethanol from cereal grains and other plant material, and the treatment of waste products comprising unwanted proteinaceous material from plant and/or animal sources, wherein the complete dissociation of the waste material is desirable.

The glutelins and prolamins in cereal grains interfere in the processing of the starch to produce ethanol, and lower the value of the protein co-product as an animal feed because of their potential to cause intolerant reactions. The present invention is to use Z. officinale rhizome to manufacture an extract or a molecule component thereof, which is capable of cleaving the glutelins and prolamins to allow the more efficient processing of the polysaccharides and to produce a protein co-product with the intolerant epitopes hydrolyzed, and consequently of a higher value as an animal feed. The invention also extends to animal feed so treated.

Accordingly, yet a further aspect of the present invention is directed to the use of Z. officinale rhizome in the manufacture of an extract or a molecular component thereof, which is capable of hydrolyzing proline-containing proteins, for degradative treatment of industrial waste products.

Examples of waste products which, in particular, may be amenable to degradation by the methods of the present invention include, inter alia, wastes from the meat and seafood processing and other food industries.

The process and cost efficiencies of ethanol production from cereals such as wheat and corn are adversely affected by proteins such as the glutelins and prolamins. The specific cleavage of these proteins by Zingibain simplifies the process of starch hydrolysis and fermentation, and adds value to the Distillers Grains and Solubles (DGS) co-product as a livestock feed.

The present invention is further described by the following non-limiting Examples.

### EXAMPLE 1

### Zingibain Protein Fraction

A protein fraction is extractable from ginger rhizome with phosphate pH6 buffer (Thompson et al., J. Food Sci. 38: 652-655; 1973; Ichikawa et al., J. Jpn. Soc. Food Nutr. 26: 377-383; 1973; Ohtsuki et al., Biochim. Biophys. Acta 1243: 181-184; 1995;). This fraction contains three closely related proteolytic enzymes that may be separated using DEAE-cellulose chromatography into two bands, GP-I containing two enzymes and GP-II containing one enzyme (Ichikawa et al., 1973, supra). These three enzymes, each with a molecular weight of about 29,000 Da, can be precipitated from the extract with acetone or ethanol leaving some small contamination from two proteins with molecular weights of 14,000 and 10,000 Da. The dominant component of GP-I has 82% homology with GP-II, and the key amino acids for proline specificity are conserved.

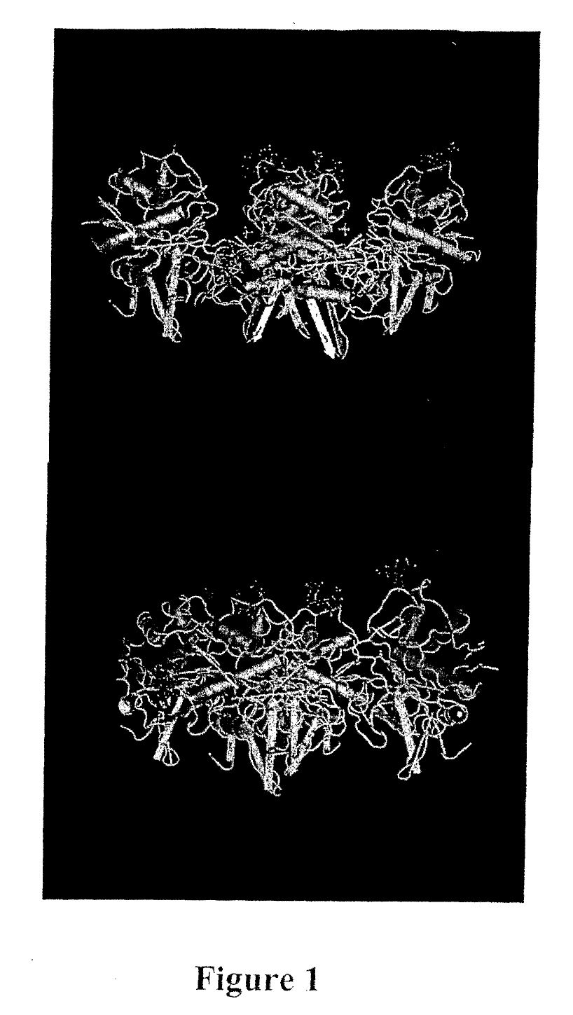

The sequence and structure of GP-II have been determined (Choi et al., 1999, supra; Choi and Laursen, Eur. J. Biochem. 267: 1516-1526, 2000). The enzyme has 221 amino acids, with the chain folded into two domains of about the same size and a cleft separating the two domains. The amino acid sequence of GP-II is set forth in SEQ ID NO:1. Domain I includes residues 13-112 and 215-218, and is mainly α-helical. Domain II includes residues 3-12 and 113-214 and has an anti-parallel β-sheet structure. This overall structure is very similar to other plant cysteine proteases such as papain and actinidin. The protein is 8% glycosylated by weight with two N-linked oligosaccharides at Asn99 and Asn156. Three disulfide bonds stabilize the GP-II protein fold. These are located between Cys24 and Cys65, Cys58 and Cys98, and Cys155 and Cys206. These residues are strictly conserved throughout the papain family. Polar residues are concentrated on the bottom of the molecule, and there is a neutral face with a radius of about 10 angstroms opposite around the active site cysteine. The active site lies in a 5.5 angstrom deep and 9.5 angstrom long cleft at the interface of the two domains. A representation of the structure of GP-II is shown in FIG. 1.

The presence of 14,000 and 10,000 Da protein contaminants in Zingibain can be explained by the self-cleavage of GP-II at Q130-P131-V132-S133, giving two fragments with 132 amino acids and 89 amino acids.

Repeated isolations give a consistent product with an activity in excess of 300 U/mg (Dionysius et al., J. Food Sci. 58: 780-784; 1993). The enzyme has also been called “proline-specific cysteine protease” (Choi et al., 1999, supra; Choi and Laursen, 2000, supra). It belongs to the Papain-like family of cysteine proteases, in which the thiol group of a cysteine is the nucleophilic group for attacking and hydrolyzing a peptide bond. This family includes enzymes such as papain from papaya (Carica papaya), bromelain from pineapple (Ananas comosus), ananain from pineapple, ficin from figs and actinidin from kiwi fruit (Actinidia chinensis).

The sequence of the dominant component of GP-I has also been determined This amino acid sequence is set forth in SEQ ID NO:2.

The term “Zingibain”, as used herein, includes the unseparated protease-containing fraction as well as isolated and purified sub-components thereof.

### EXAMPLE 2

### Hydrolysis of Collagen by Zingibain

Collagen is the most abundant protein in humans, accounting for about 25% of protein, and its structure is largely conserved in the animal kingdom from the most primitive animals to humans. It is expressed in fibroblast cells. It forms the organic mass of skin, tendon, blood vessels, bone, the cornea, vitreous humor of the eye, and basement membranes. It polymerizes into a triple-stranded helix with each strand over 1,000 amino acids. The major form of collagen in most species is designated as collagen I and has two α1(I) chains and one α2 chain, [α1(I)]₂α2. Cartilage collagen has the structure [α1(II)]₃, and collagen that occurs in various tissues, especially embryo tissue, has the structure [α1(III)]₃. Collagen is very rich in proline and hydroxyproline. Collagen proteins have an extremely high number of sites with the right combinations of amino acids for hydrolysis by Zingibain, but the three-dimensional structure especially the tight helical structure limits the sites where hydrolysis can take place.

An azocollagen (azocoll) assay was used to study the hydrolysis of collagen with Zingibain. The azocollagen (Sigma Aldrich) substrate suspension was prepared by mixing 0.1 g washed and ground azocollagen powder with 10 mL of assay buffer (0.1 M sodium phosphate pH 6.0 containing 1 mM DTT and 1 mM EDTA) in a small measuring cylinder on a magnetic stirrer at room temperature. After 30 min, 1 mL of the suspension was transferred with a wide bore micropipette (diameter 2.5 mm) to a glass test tube (150 mm×13 mm) without depositing any of the suspension on the walls of the tube. The tube was equilibrated for 5 min at the designated temperature in a shaking water bath having a horizontal displacement of 4 cm at a speed of 112 passes per min. The enzyme sample (50 μL) was incubated with the substrate for 30 min with constant shaking in the water bath. The reaction was stopped with 1 mL cold 10% v/v trichloroacetic acid (TCA) and the reaction mixture transferred to a 2 mL microfuge tube. After centrifugation at 12,000 rpm for 5 min, the supernatant was removed and its absorbance read at 520 nm. A sample blank was prepared by incubating 1 mL substrate for 30 min, adding 1 mL 10% f/v TCA and then 50 μL enzyme sample.

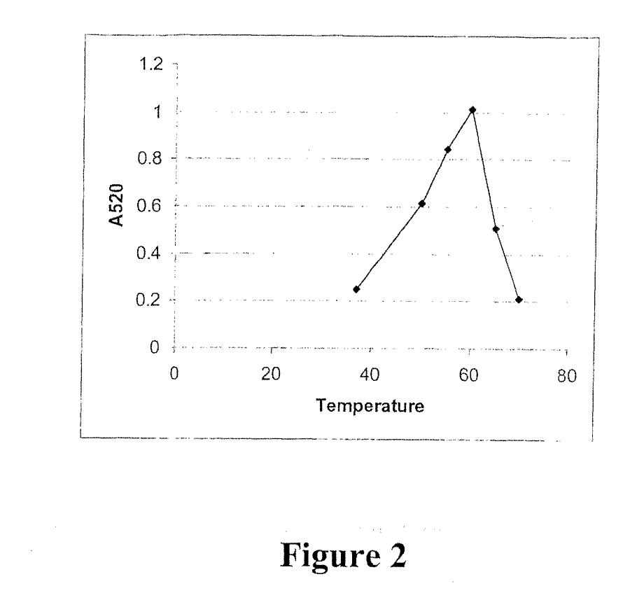

The studies showed that the reaction did not conform to Michaelis-Menten kinetics. The absorbance increased with substrate concentration linearly up to the maximum achievable concentration of 5%. The effect of temperature showed an increase in rate above 50° C. with a maximum rate at 60° C. about four times the rate at 37° C., and with the rate falling off sharply so that at 70° C. the rate was reduced to one-fifth the maximum rate. The increase in absorbance at 60° C. was found to be linear with time up to 90 min. The reaction rate was relatively linear with enzyme concentration over the range of 25-500 μg Zingibain. These results are set forth in FIG. 2.

An SDS-gel electrophoresis study of the hydrolysis of beef-muscle collagen showed that Zingibain was effective at breaking down collagen up to 70° C. The fragmentation pattern differed from that for ficin and papain, and from that for Clostridium histolyticum collagenase which produced large fragments of collagen over 3.5 days whereas Zingibain fully degraded the collagen to low molecular weight fragments of the order of 2,000 Da and less. The study showed Zingibain attacked the helical section of collagen. This region would become more susceptible to hydrolysis as the helical structure was loosened.

Hence, treatment of meat for feed/food with Zingibain rapidly yields fully degraded collagen, making such treatment ideal for use in tenderizing meat for animal consumption.

Another SDS-electrophoresis study of acid-soluble Type I collagen showed that the triple-stranded γ-form of collagen was attacked at low Zingibain levels forming a band not far below the γ-band, with lines also appearing below the bands for the single-stranded oil and α₂ collagen, and with bands for the double-stranded β form and its degradation products. As the concentration of Zingibain was increased, the γ- and β-bands quickly disappeared with only weak α-bands still visible with clear sharp bands of degradation products at lower molecular weights than the 100,000 Da α-form. At higher concentrations of Zingibain, only bands due to Zingibain and low molecular weight fragment products were visible. These observations also support the proposition that Zingibain attacks the helical regions and not simply at the telopeptides at either end of the molecule, as these sections are too small to account for the fragmentation patterns observed.

The controlled hydrolysis of proteins in foods such as collagen in meat and seafood during the cooking process allows protocols to be developed for more efficient preparation of highly palatable food with shorter cooking times and less energy expenditure. Cooked corned silverside with a regular commercial protocol of a 25% pump with total salt in the meat at 2% of final weight is given as an example using the ‘outside flat’ muscle (M. biceps femoris). Normally, corned beef is cooked for a long period of time to achieve an acceptable degree of tenderness, but using the ginger extract the desired tenderness was achieved at low temperature and short cooking times with, if necessary, a higher temperature spike to achieve the desired degree of doneness: Short cooking times (time is dependent on size of piece of meat) in a water bath at 75° C. with 15 mg Zingibain per Kg beef produced a tender corned meat with a shear force and compression of less than 3.5 Kgf and 1.1 Kg, respectively, compared to 4.3 Kgf and 1.7 Kg for meat with no Zingibain added for a 250 g piece of meat cooked for up to 60 minutes with a small Cooking Loss of less than 6%. To achieve that tenderness without Zingibain, much longer cooking times are required with the associated higher Cooking Loss.

### EXAMPLE 3

### Hydrolysis of Prion Proteins by Zingibain

Proteinaceous infectious agents, known as “prions”, have been identified and characterized over the past two decades. These infectious agents are known to be the causative agent in the spongiform encephalopathies, such as:
Bovine Spongiform Encephalitis (BSE), or Mad Cow Disease;
Scrapie, the disease of sheep and goats;
Creutzfeldt-Jakob Disease of humans, of which 10-15% of cases are due to heritability while some cases are caused inadvertently through operation infection, and perhaps through blood transfusions;
Gerstmann-Straussler-Scheinker Disease; and
Fatal Familial Insomnia.

They consist mainly, if not exclusively, of a protein called prion protein (designated PrP). It is known that one form of PrP causes the disease (PrPsc), and a second form (PrPc—the normal form) does not.

(a) Prion Structure

The difference is apparently caused by a conformational change in the protein structure. Normal PrPc consists primarily of α-helices, and the diseased PrPsc consists primarily of β-sheets. Apparently, the presence of the PrPsc can cause the normal PrPc to change conformation and become the infectious PrPsc. It is further thought that, for humans or other animals carrying a mutated gene, the mutation may render the PrPc susceptible to flip from the α-helix to the β-sheet conformation. This change takes time to occur, as does the accumulation of enough infectious PrPsc to damage the brain sufficiently to cause symptoms.

(b) Proline Prevalence and Zingibain Susceptibility

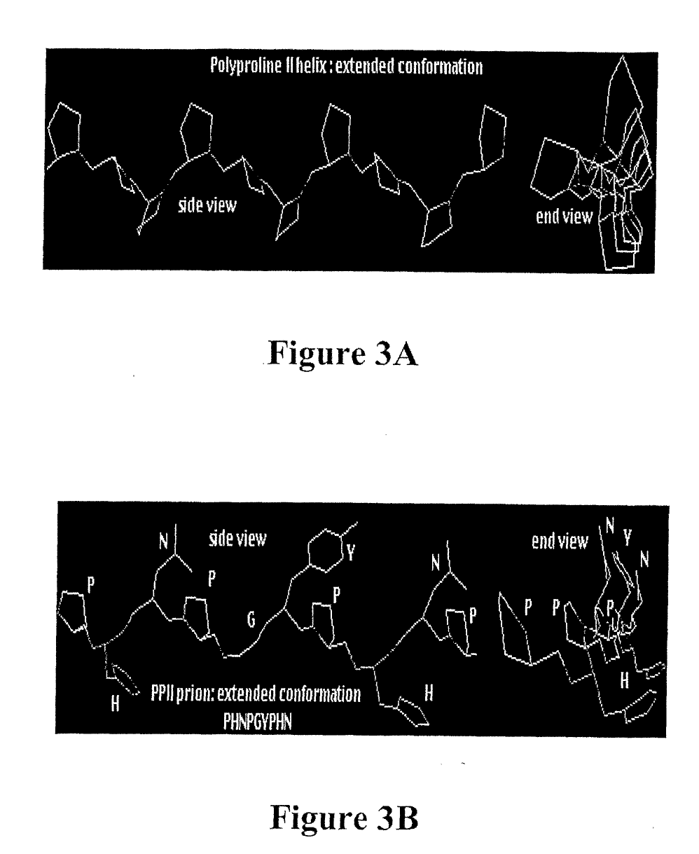

The structure of prions shares some similar features to collagen, including the presence of a repeat region that contains proline in an amino acid unit that is repeated. In chicken prion, for example, there is a 54-amino-acid region with nine repeat units (PHNPGY) [SEQ ID NO:4] in which proline is every third amino acid, thereby forming an extended polyproline II helix (refer to FIG. 3), as is also found in collagen.

Normal PrP is protease-sensitive. However, PrPsc in infected brains resists breakdown with proteases. Given the structure of prions, however, they represent ideal targets for hydrolytic degradation by Zingibain. Zingibain, having proline-rich natural proteins as its preferred target molecule, may render PrPsc harmless through proteolytic cleavage. Bovine prion is a 28,600 Da protein, having the sequence seen below (also set forth in SEQ ID NO:3):
1 MVKSHIGSWI LVLFVAMWSD VGLCKKRPKP
31 GGGWNTGGSR PGQGSPGGN RYPPQGGGG
60 WGQPHGGG* WGQPHGGG* WGQPHGGG*
84 WGQPHGGG* WGQPHGGG*
100 GWGQGGTHGQ WNKPSKPKTN MKHVAGAAAA
130 GAVVGGLGGY MLGSAMSRPL IHFGSDYEDR
160 YYRENMHRYP NQVYYRPVDQ YSNQNNFVHD
190 CVNITVKEHT VTTTTKGENF TETDIKMMER
220 VVEQMCITQY QRESQAYYQR GASVILFSSP
250 PVILLISFLI FLIVG

The 18 prolines are indicated in bold face “P”. Of these, 16 have a hydrophilic residue preceding or following the “P”. The 5 prolines in the repeat units “WGQPHGGG” [SEQ ID NO:4], indicated with an asterisk, thus: *, and Pro53 have glutamine adjacent to the proline; Pro28, Pro41, Pro148, and Pro176 have arginine; Pro30, Pro113, and Pro116 have lysine adjacent to them; Pro169 has asparagine; and Pro46 and Pro249 have serine.

All of these prolines, if exposed to attack by Zingibain, are susceptible to hydrolysis. Moreover, the prions are universally structured for multi-site hydrolysis by Zingibain, exactly as is collagen. As the more exposed regions are cleaved, the internal structure is exposed for hydrolysis, so that under the appropriate conditions, the protein may be completely destroyed.

(c) Prevention of Prion Transmission Through Blood Transfusion

One possible life-threatening event that is prevented through the application of Zingibain is the unexpected/undetectable transmission of infectious prion protein through, for example, blood transfusion. PrPsc can be transmitted to blood recipients and cause disease by interacting with PrPc molecules, inducing them to change conformation to the disease-causing β-sheet-prevalent PrPsc structure. The possible transmission of Spongiform Encephalopathies through blood transfusion is a major concern because the prion would be very difficult to detect, and the disease takes many years to produce symptoms. Prior treatment of blood to be transfused with Zingibain may obviate this dangerous possibility. Similarly, surgical equipment and blood-processing equipment may be decontaminated by Zingibain if exposed to prion molecules, preventing the transmission of the disease.

(d) Preparation of Prion-Free Food and Feed

Meat and meat products, prepared for both the human food and animal feed markets, may be routinely treated with Zingibain to cause the degradation of potentially-fatal prions, rendering the to-be-consumed product prion-free and hence safe.

### EXAMPLE 4

### Zingibain-Eradication of Allergenicity and Food Intolerance

Many commercially important plant proteins are proline rich. Even the pollens collected by bees from Australian native trees are particularly rich in proline: the amino acid Pollen Analysis for eight common pollen sources in 1990/91 gives the following mean values, in percentage (Stace, “Protein Content and Amino Acid Profiles of Honeybee-Collected Pollens” Bees‘N’Trees Consultants, Lismore, NSW, Australia, 1996, 2480):

Threonine 3.51 Leucine 6.25 Lysine 5.90

Valine 4.70 Isoleucine 3.83 Histidine 2.13

Methionine 1.75 Phenylalanine 3.75 Arginine 5.3

Tryptophan 2.65 Aspartic acid 8.62 Serine 4.43

Glutamate 10.36 Proline 11.69 Glycine 4.23

Alanine 5.00 Cystine 0.84 Crude protein 22.7%

In instances where these pollens are allergenic, Zingibain removes the allergens through protein hydrolysis.

### EXAMPLE 5

### Removal of Gluten Response in Person with Celiac Disease

A person with a well established history of symptoms of celiac disease, who had been on a strict, long-term “gluten-free” diet was provided with a daily intake of various products made from wheat flour that included, in the ingredients, the filtered ginger crush product. The products included buttercake, ‘devilishly dark chocolate torte’, French bread, egg pasta (spaghetti), commercial whole wheat breakfast biscuits and commercial bread. The non-commercial products were prepared as follows:

(a) Buttercake

An oven was pre-heated to 180° C. A cake tin was brushed with melted margarine, and the base lined with baking paper. Using an electric beater, 125 g margarine and % cup castor sugar were beaten in a small mixing bowl until light and creamy. Two eggs, lightly beaten, were added gradually, beating well after each addition. One teaspoon of vanilla essence was added, and the mixture beaten well until combined.

The mixture was transferred to a large bowl. Using a metal spoon, 2 cups of sifted self-raising flour were folded in, alternatively, with ½ cup milk. The mixture was stirred until just combined, and 1 teaspoon of filtered ginger crush solution was added and the mixture again stirred until almost smooth.

The mixture was spooned into the prepared tin, and the cake baked for 45 mins, when a skewer inserted into the centre of the cake came out clean. The cake was left in the tin for 10 mins, and then turned onto a wire rack to cool.

(b) ‘Devilishly Dark Chocolate Torte’

The base and sides of a deep 23 cm square cake pan were greased with margarine and the base of the pan was covered with baking paper.

Margarine (185 g) was melted in a saucepan, and removed from the heat. One cup of double-strength short-black coffee was stirred in, combined with 150 g chopped dark chocolate and ½ cup castor sugar, and the mixture stirred until smooth. The mixture was placed in a large bowl of an electric mixer. To this was beaten-in, in three batches, a sifted mixture of 1 cup self-raising flour, % cup plain flour, and 2 tablespoons cocoa, followed by 2 eggs, 1 teaspoon vanilla essence and 1 teaspoon filtered ginger crush Zingibain solution.

The mixture was poured into the prepared pan, and baked in a slow oven (150° C.) until firm (1.25 hr). The cake was stood for 5 mins before being turned onto a wire rack to cool.

The cake was cut in half, and each half split into three layers. A layer of cake was placed on a serving plate and spread thinly with raspberry jam. A thin layer of a filling was then made by combining 200 g hot melted dark chocolate and 125 g margarine in a bowl, stirring in ¼ cup sifted pure icing sugar, cooling to room temperature and beating with a wooden spoon until the filling was thick and spreadable. This was topped with another layer of cake, which was sprinkled with a little Creme de Cacao, then spread thinly with the filling. The layering was repeated with the remaining cake, liqueur and filling. The layered cake was refrigerated for several hours until it was firm.

Filling (⅔ cup), which had been reserved, was spread evenly over the cake.

(c) ‘French Bread’

A Breville Master Excel Bread and Dough Maker was used with its recipe (except for the added filtered ginger crush Zingibain solution) for ‘French’ Bread (750 g loaf), with the following ingredients added in the set order: 310 ml water; 1.5 teaspoons filtered ginger crush Zingibain solution; 2 teaspoons extra virgin olive oil; 1.5 teaspoons salt; 2 teaspoons sugar; 3 cups (450 g) unbleached plain flour (12% protein); 1 teaspoon Bread Improver; and 1.5 teaspoons dry yeast.

A medium setting was used, which had the following program: 1^{st} knead; 2^{nd} knead; 1^{st} rise; punch down; 2^{nd} rise; punch down; 3^{rd} rise; bake for a total time of 3.36 hr. The bread rose to close to the top of the container.

(d) Egg Pasta (Spaghetti)

A Breville Master Excel Bread and Dough Maker was used with its recipe (except for the added filtered ginger crush Zingibain solution) for Egg Pasta Dough, with the following ingredients added to the bread pan in the set order: four lightly beaten eggs (60 g); 1.5 teaspoons filtered ginger crush Zingibain solution; 1 tablespoon extra virgin olive oil; 1 teaspoon salt; 2 cups (300 g) plain flour; 1 cup (170 g) semolina.

Pasta dough setting of “8” was used with a processing time of 13 minutes. The dough was rolled into a cylinder using a buckwheat “gluten-free” flour dusting on the plastic sheet. It was cut into portions and put through a pasta maker to prepare spaghetti, which was allowed to dry for 1 hr prior to packaging.

In week 1, the person ate one slice of the buttercake each afternoon. In week 2, the person ate one slice of the chocolate cake each afternoon. In week 3, the person ate two slices of the bread for lunch each day, and on the third day ate a dish of the spaghetti for dinner.

The person monitored closely any response of her body to the food. There was no sign of any adverse effect. Considering that her normal adverse response time, after eating a product with wheat flour, was about 2 hr before diarrhoea and flatulence set in, the lack of any sign of response, after consuming the above range of wheat flour products, was seen as evidence supporting the conclusion that the ‘gluten’ intolerance epitopes of gliadin and glutenin had been removed by the ginger crush Zingibain solution.

(e) Whole Wheat Breakfast Biscuits

‘Washed’ wheat was conditioned with water containing ginger crush solution at the rates of 1.6 mL crush per Kg wheat and 16 mL per Kg wheat and processed commercially to make standard whole wheat biscuits. The person was provided with two sets of two biscuits: treated with 1.6 mL crush, and treated with 16 mL crush, and asked to eat them dry and record response. The person had no adverse response to the treated biscuits, and found them very palatable.

(f) Commercial Bread

A series of batches of bread made from 3 Kg wheat flour containing 1900 mL water were made in a commercial bakery with increasing amounts of the ginger crush added to the water. From the lowest level of ginger crush (1 mL) to 120 mL of ginger crush added, the dough took less time in the mixing (10 minutes reduced to 8 minutes) to reach maturity and was judged by the baker to be finer and to cut better than the dough with no crush added, and the bread in a closed tin and in an open tin was larger with the crust more uniform, the bread whiter and with a finer texture. The treated bread had a longer shelf life. The bread was able to adsorb the additional 120 mL of water without any deleterious effects. To maintain this quality with 240 mL crush, the water had to be reduced by 120 mL.

Sets of two slices of each batch of treated bread were packaged and frozen, and delivered with instructions to eat the two slices per day starting from the highest number written on the package (No. 9) (240 mL crush) and proceeding to the lowest number (No.: 2) (1 mL crush). The person had no adverse intolerant reaction to any of the batches.

The person has continued to eat cereal products made with the ginger crush solution for over a twelve month period without an adverse response, but the person's adverse response to food containing normal ‘gluten’ remains severe.

The above set of tests has been repeated with seven other people with well defined symptoms of gluten intolerance including Coeliac Disease and Dermatitis herpetiformis with no adverse response to the various types of wheat based foods when treated with the ginger crush solution.

The gluten proteins were extracted from the above bread samples with 65% ethanol and studied by SDS-PAGE electrophoresis. Compared to the standard loaf of bread, the gluten proteins in the treated bread had been made more soluble by the ginger extract with each protein band being more intense for an identical extraction and electrophoresis. The bands for the high molecular weight and low molecular weight glutenin units and for the gliadins were shifted to lower molecular weight. As the level of extract was increased, the bands became more diffuse possibly due to multiple site hydrolysis. These results are consistent with the zingibain clipping the very high molecular weight glutenin conglomerate structures to release higher concentrations of the gliadins and glutenins under 100,000 Da with the molecular weights of the detectable gliadins and glutenins shifted to lower values consistent with the observed removal of the gluten intolerance.

### EXAMPLE 6

### Improved Quality of Food for Human Consumption

The effects of Zingibain on wheat, corn and oats have been examined in terms of the quality of food consumed by humans. The effects were dramatic, as reported by standard “taste test” panels of experts.

(a) Coffee Buns

Coffee buns were made from wheat flour mixed with low-fat milk, one batch containing Zingibain and the other without. The mixture was left overnight at about 37° C. The two doughs were significantly different: the Zingibain-treated dough was more like a pliable plastic. When mixed with margarine, sugar, eggs, sodium bicarbonate and egg and cooked at 200° C. for 15 minutes, the buns were both pleasant to eat. However, in a blind taste-test with experienced cooks, the Zingibain-treated buns were selected as being light, having a good front-palate and a silken after-palate, and having no hint of a “soda-flour” taste that is characteristic of “scones” and as was found for the other buns.

(b) Bread

Bread made from wheat flour, which had been sifted with Zingibain and included in a standard milk bread mix, yielded a product that was selected by the taste panel as far superior to the bread made without the Zingibain, with a structure that was much finer and more uniform.

(c) Crepes

Cornmeal crepes were made from “Mellow Yellow” polenta in a standard recipe with Zingibain sifted with the corn flour ingredient for one batch, and the flour sifted without Zingibain for the second batch. The two batches of batter were left at 37° C. for one hour prior to cooking. A trained cook prepared the crepes (“blind”) and reported during the cooking that the two batters were very different, with one staying on the crepe pan exceptionally well and the other displaying characteristics of a typical cornmeal crepe batter, which is always difficult to keep on the pan. Again, when the two batches of crepes were tasted ‘blind’, the difference was very marked. One batch was reported to be very smooth and uniform with no grittiness, whereas the other was typical of cornmeal crepes with grittiness and inconsistent structure. The panel commented that it was the first time they had eaten a cornmeal crepe that they had enjoyed.

(d) Oats Porridge

Oats were cooked as porridge in the standard way, with the exception that cool tap water was added initially, instead of boiling water. One batch contained Zingibain. In contrast to the batch with no Zingibain, the Zingibain porridge had no firm grains: all the grains had become gelatinous.

(e) Gluten-Safe Products of Example 5

The buttercake, devilishly dark chocolate torte, French bread, egg pasta (spaghetti) commercial whole wheat breakfast biscuits, and commercial bread food items, prepared in accordance with the experiment set forth in Example 5, above, were also classified as extremely palatable by the people undergoing the trial.

The commercial bread trial with the ginger extract added in Example 5f produced satisfactory bread with up to 4% additional water, with the dough mixing time reduced from 10 minutes (basic formulation) to 8 minutes, with up to 20% increase in height ex prover, and up to 3.8% increase in height ex oven, with improved colour and texture as measured by eye, with the crumb colour (measured 2 days after baking) improved by 2.2%, and the bread texture (measured 2 days after baking) measured in Newtons reduced from 3.15 (basic formulation) down to 2.14.

These examples show that the inclusion of Zingibain in cereal grain food items during preparation results in a more palatable and preferred product.

### EXAMPLE 7

### Improved Quality of Feed for Animal Consumption

Companion and commercial animal feeds contain proteins from a broad range of sources such as cereals, soy, cottonseed meal, and animal by-products. Enzymes in animal feeds improve the nutritive value of foodstuffs and reduce pollution as a consequence of better utilization of feed. All animals use digestive enzymes that are produced by the animals themselves or by the micro-flora of the gastrointestinal tract, but the feed-conversion efficiency is not 100%. For some animal/feed combinations, up to 25% of the feed is not digested.

Exogenous enzymes are therefore used to break down anti-nutritional factors such as lectins and trypsin inhibitors and allergenic and intolerance epitopes that are present in many feed ingredients and that are not broken down by endogenous enzymes. These can otherwise interfere with normal digestion, causing poor performance and, food intolerance reactions with associated auto-immune diseases. Exogenous enzymes also increase the availability of carbohydrates, proteins and minerals, which are either enclosed within particularly resistant cell walls, and therefore not as accessible to the endogenous enzymes, or are bound up in a form that the animal cannot digest. They also break down specific chemical bonds in raw materials, which that are not usually broken down by the endogenous enzymes, thereby releasing more nutrients.

Young animals, in particular, benefit from exogenous enzymes because of the immaturity of their own digestive system. (“The current feed enzyme market and likely trends” in “Enzymes in Farm Animal Nutrition” Bedford, M. R.; Partridge, G. G. (eds) CABI Publishing Marlborough UK, 2001). Furthermore, the laying-down of meat muscle cells at the time of weaning makes it imperative to provide the weaner with pre-digested protein-rich feed. The normal raw materials for this are skimmed milk powder, whey powder and derivatives, blood and blood plasma products, processed fishmeal, “low antigen” soy proteins, and cooked cereals.

Although the main storage proteins of soybeans, glycinin and β-conglycinin, are implicated in changes to the intestine of young pigs fed with this legune (Li et al., J. Animal Sci. 69: 4062; 1992), it will continue to be included in feeds because of its high protein level and low cost. The antigenicity of these proteins is removed by cleaving the antigen epitope, through pre-digesting the feed with Zingibain.

The trypsin inhibitors of soy, unless destroyed before feeding, will cause the pancreas to produce protein-rich secretions with concomitant loss of cells lining the gut (Partridge, “Considerations in the Formulation of Piglet Creep and Starter Feed” Technical Bulletin, American Soybean Association, 1997). Prior incubation with Zingibain also causes reduction of trypsin inhibitors, through hydrolysis.

A solution of Zingibain is mixed with the protein source at room temperature or at temperatures up to 65° C. to pre-digest the feed protein before it is added to other ingredients and pelletized. The latter process sometimes occurs at a higher temperature, where Zingibain is then deactivated. Alternatively, Zingibain is added to the dry feed either in its active state as a dry powder, or as a solution, just prior to being fed to animals. In addition to improving feed efficiency, Zingibain improves overall animal health.

### EXAMPLE 8

### Trials of Zingibain in Dog Food

The following trials were conducted using a commercial “Super Premium Adult Active” dog food.

The following results show that, although it is a “Super Premium” dog food, it did not have the healing power or Metabolisable Energy as the food plus Zingibain powder. The crude protein is guaranteed at 30%, derived mainly from corn, chicken meal and dried egg product.

Dogs were housed in 24 square metre secure, concreted individual enclosures with a total of 18 square metres under cover and with a 6 square metre insulated internal night kennel. The staff feeding and monitoring the dogs did not know when Zingibain was introduced into the feed, or when the level of Zingibain was changed.

(a) Trial 1

The aim of this trial was to reduce arthritic pain from a major bilateral hip joint abnormality, and to increase weight to an appropriate level for the breed, age and gender, taking into account the hip problem.

The dog tested is a 2.5-year-old Airedale Terrier bitch, who was about 7 kg under standard weight (weight: 18.3 kg; standard weight: 25 kg), even though she was on a higher than normal daily feed intake (400 g Adult Active; normal for 20-30 kg active dog is 260-360 g). Because of inflammation of the hip joints, the dog was finding it increasingly difficult to get up in the morning and to be her normal active self.

Zingibain powder (32 mg) was mixed daily, with the 400 g Adult Active dry feed, starting on Day 1. For the period Day 33 to Day 40, only 16 mg Zingibain was added, to see if a lower level had a lesser effect. The dog was monitored regularly in order to determine any effects of the Zingibain on the dog's health and behaviour. Her faeces were collected and weighed wet daily, and each week's collection was air dried separately to determine any changes to Digestibility Percentage. The dog was weighed and inspected by a veterinarian on Day 0 and then from time to time during the trial.

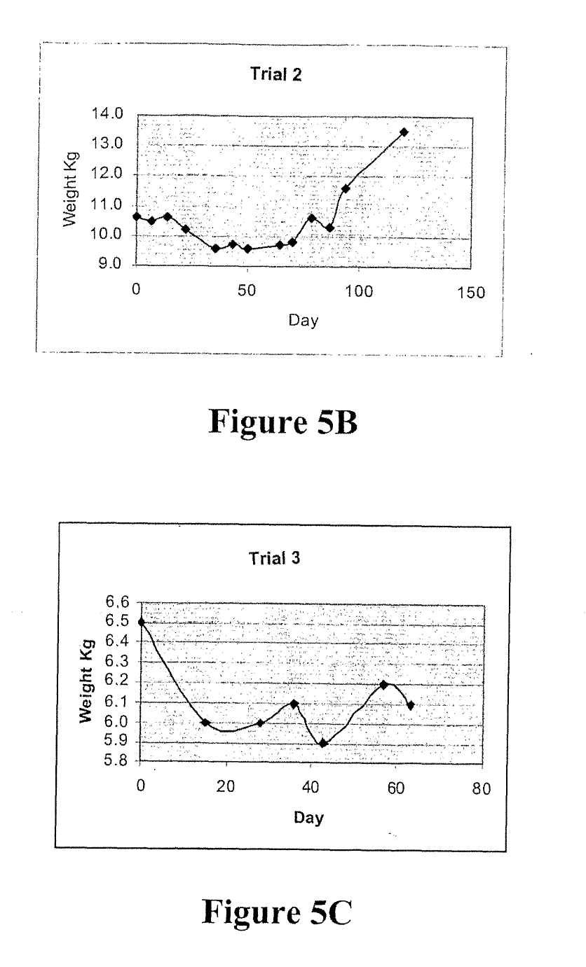

The dog rapidly showed behavioural changes with no obvious signs of arthritic pain, although the looseness in the hip joints was obvious when she ran. She became much more active in her general behaviour, with no reluctance to get up in the morning. The dog's weight changed as shown in FIG. 5A, from 18.3 kg on Day 0 to 23.3 kg by Day 87. The reduction in the level of Zingibain from Day 33 to Day 40 affected the weight gain, and when a bitch in an adjacent kennel came into season on about Day 57, and the dog came into season on about Day 78, this also impacted on the weight gain. Her Digestibility Percentage (100[weight feed−weight dry faeces]/weight feed) increased from 79% to about 82% over the 87 days. The dog's average daily weight gain of 57.5 g from her 400 g feed greatly exceeded the 12 g per day expected from the decrease in faecal weight.

Therefore, Zingibain supplementation to feed is able to affect the general health of animals, by reducing arthritic pain from severe joint abnormalities. It is further able to increase the feed's Metabolisable Energy significantly, to allow an animal to gain weight even though the same feed and level of feed (higher than normal) had been eaten for 12 months without a significant change in the animal's weight.

(b) Trial 2

In this trial, the aim was to stop major, rapid weight loss and bleeding from the anus, and to restore the health and weight of the dog. The veterinary diagnosis was that the bleeding was from either multiple gastrointestinal ulcers or cancer, with a possible secondary tumour in the liver causing the severe weight loss.

The dog tested is a 5-year-old Kerry Blue Terrier dog whose normal weight had been about 17.5 kg, which is close to the standard for the breed, age and gender. However, although he continued to eat his 200 g Adult Active feed daily, as he had for the previous 12 months, he suddenly lost weight (about 7 kg) over a few weeks and blood was noted in his faeces and, later, severe anal bleeding was observed.

Zingibain powder (16 mg) was added daily to 200 g Adult Active dry feed from Day 4 to Day 21. The level of Zingibain was increased to 32 mg from day 22 to day 39; it was reduced to 16 mg for Day 40 to Day 47, when it was increased again to 32 mg per day to see if there was any dependence on the Zingibain level.

From Day 0, the dog's faeces were inspected for any sign of blood, and its anus was wiped with a tissue to look for blood. From Day 8, the faeces were collected daily and weighed, and each 7 day collection was held separately and air dried. The dog's general health and behaviour were monitored closely each day, and he was weighed and inspected by a veterinarian from time to time during the trial.

Within 24 hr of adding Zingibain to the diet (on Day 5), bleeding ceased. No evidence of blood was observed in the faeces or on the tissue and no evidence of bleeding has been found since. Although the open would had been healed sufficiently to stop bleeding, the underlying disease and associated weight loss took longer to be controlled. The dog continued to eat his whole diet each day, and he showed improved health with plenty of energy. His weight loss slowed down and his weight bottomed at 9.6 kg on Day 35, and commenced increasing on Day 64 with a dip at about Day 87 when the bitch in the next enclosure came into season. By Day 120, the dog's weight had increased by about 4 kg from his minimum (refer to FIG. 5B). The dry faecal weight showed no significant trend.

In this instance, Zingibain supplementation to dog food assisted in curing gastrointestinal disease that causes severe blood and weight loss.

(c) Trial 3

The aim of this trial was to provide a dog's Maintenance Energy Requirement by administration of 75% of the normal feed, to which was added a Zingibain supplement.

The test dog is a 7-year-old Miniature Schnauzer bitch. She was in excellent condition, at 6.5 kg (standard weight is 6.0-6.5 Kg). She had been on Adult Active feed for over 12 months, at 100 g per day. For the duration of the trial, the dog was fed 75 g Adult Active dry feed (75% normal feed). On Day 4, Zingibain powder was mixed with the feed: Zena received 8 mg for Day 4 to Day 32, 4 mg from Day 33 to Day 40, 8 mg from Day 41 to 57, 0 mg from Day 58. She was returned to her normal diets on Day 64, when her condition started to be affected by the reduced diet with no Zingibain.

The dog was monitored regularly; her faeces were weighed as above, and she was weighed and inspected by a veterinarian from time to time, as were the other animals in the trials.

For the first three days, when the feed level was reduced without Zingibain, the dog lost weight. However, no further weight was lost when the Zingibain was added, and the weight actually started to increase. When the level of Zingibain was reduced to a half, she lost weight again, but recovered her weight when the level of Zingibain was again increased. When the Zingibain was removed from the feed, the dog showed a loss of condition and a weight loss to the extent that it was deemed necessary to terminate the trial after Day 63 because of the loss of general condition (refer to FIG. 5C).

A similar set of results was obtained for another dog.

It is, therefore, apparent that significantly lower levels of feed can be used to achieve the Maintenance Energy Requirement of a dog, if about 1 mg Zingibain per kg dog weight is added to the feed.

### EXAMPLE 9

### Trials of Zingibain in Chicken Feed

In this experiment, the aim was to determine the effect of treating commercial chicken feeds with a solution of Zingibain (1 mg Zingibain per kg chicken, in the average food eaten per day) on the live weight gained and on a number of other parameters as follows: carcase weight, breast meat yield, total protein, fat and ash, and palatability of the meat.

Newly hatched chickens were purchased for the trial. Most were initially a black/grey colour, some with yellow dots. Others were yellow, some with black dots, and there was one brown chicken. They were sufficiently different in their colour patterns so that identification during the trial was possible. Chickens of the same general colour were put in boxes and were divided by ‘blind selection’ into two indoor pens 1 m×2.5 m, one for control and one for treated chickens, each with an adequate water supply and four feeding trays. The single brown chicken was put into the “red” pen, which was the pen for the Zingibain treatment. Each chicken was weighed at about 11:30 a.m. on (Day 1), about 2 hours after hatching. Subsequently, chickens were weighed each day at about 7:00 a.m. and 5:00 p.m., and after day 20, the weight of feed remaining uneaten in each pen was weighed twice a day at these times. Each pen had a 60-watt bulb light set at an appropriate distance from the floor to ensure the chickens were kept warm. The chickens were free to eat 24 hr each day (ad libitum) from four feed trays.

Commercial feeds used in the trial were purchased from local produce agents. The feeds ranged in protein content from 14 to 20%. The feeds were treated with water, and Zingibain was added to the trial feed to give 2 mg Zingibain per kg chicken per day.

The chickens were coded and, after they reached the targeted weight range, were processed. Twenty frozen carcases (five control hens, five control roosters, five trial hens and five trial roosters with live weights and average live weights matched) were analyzed for breast meat yield, meat quality and cook loss. Statistical analyses of all the data were then undertaken.

Pairs of control and trial carcases, matched for weight, were selected from the coded chickens by an independent person, and the chickens were roasted side by side in a fan-forced convection oven in separate trays (Trial 1) or in pierced oven bags (Trial 2) at 200 C (Trial 1) or 175 C (Trial 2). The circumference of each drumstick muscle was measured before the birds were cooked to the same degree of doneness and the meat analyzed by a tasting panel. The data for the complete feeding trial are set forth below.

TABLE 2
Predicted means for the effect of Zingibain on final live-weight
and carcass weight after adjustment for sex and after
correction to the same initial live-weight

| Trait (g) | Control | +Zing | Se* | Prob* |
|---|---|---|---|---|
| Final live-weight | 2223.9 | 2342.7 | 70.0 | 0.2342 |
| Plucked weight | 2095.5 | 2208.4 | 68.1 | 0.245 |
| Carcass weight | 1527.7 | 1630.2 | 48.9 | 0.1439 |

*Se standard error; Prob: probability of pairs of results being identical

The final live-weight, plucked weight and carcass weight all showed a trend for increased weight in the Zingibain supplemented group (+Zing).

TABLE 3
Predicted means for the effect of Zingibain on breast
weight, pH, cook loss, drip loss, peak force and composition,
after adjustment to the same carcass weight

| Trait | Control | Zing | Se* | Prob* |
|---|---|---|---|---|
| Breast weight g | 256.3 | 272.4 | 7.1 | 0.128 |
| pH | 5.37 | 5.37 | 0.04 | 0.96 |
| Cook loss (%) | 19.8 | 18.3 | 0.01 | 0.294 |
| Drip loss (%) | 8.2 | 7.5 | 0.9 | 0.612 |
| Peak force kg (cooked) | 1.86 | 1.89 | 0.12 | 0.951 |
| Chem Fat %: | | | | |

| | | | | |
|---|---|---|---|---|
| Breast | 8.47 | 7.93 | 0.39 | 0.342 |
| Remainder | 27.24 | 26.51 | 0.51 | 0.321 |
| Body | 23.43 | 22.41 | 0.54 | 0.203 |

| | | | | |
|---|---|---|---|---|
| Ash (%) | 6.46 | 6.43 | 0.21 | 0.816 |
| Protein (%) | 3.93 | 4.72 | 0.37 | 0.154 |
| Dry Matter (%) | 33.7 | 33.6 | 0.46 | 0.799 |

*Se standard error; Prob: probability of pairs of results being identical

Zingibain treated chickens had 16 g (6%) more breast muscle weight than the controls when compared at the same carcass weight. The same trends were found for the three individual muscles of the chicken breast. Using the full data set in the first of these tables, there was a trend for the Zingibain treated chickens to produce heavier carcasses than the controls. These means were then used to calculate the breast weight for Zingibain-treated and control chickens, using the regression equations calculated from the data from the 20 frozen carcasses referred to above. The predicted breast weight of a Zingibain-treated chicken with a carcass weight of 1630.2 g was 269.8 (+/−7.1), whilst the predicted breast weight of a control chicken with a carcass weight of 1527.7 g was 236.0 g. From these data, the cumulative advantage of Zingibain treatment was estimated to be of the order of 14%.

There was no effect of Zingibain treatment on pH or shear force for the cooked breasts, reflecting the lack of collagen in chicken breast. The cook and drip loss showed a trend for lower losses in Zingibain treated samples. Chemical fat percentage was measured on the breast and the remainder of the carcass (skin off) showing a trend to lower fat in the treated chickens. Protein percentages trended to a higher percentage for the treated chickens.

TABLE 4
Cooked meat taste tests

| | Trial 1 | | | | Trial 2 | | | |
|---|---|---|---|---|---|---|---|---|
| | Sex of chicken: | | | | | | | |
| | Control: M | | Test: F | | Control: F | | Test: M | |
| Drumstick circumference | 14.1 | cm | 14.7 | cm | 15.7 | cm | 16.7 | cm |
| Carcass weight uncooked | 1482 | g | 1518 | g | 1850 | g | 1900 | g |
| Carcass weight cooked | nm | | nm | | 1295 | g | 1370 | g |
| Carcass cook loss | nm | | nm | | 555 | g | 530 | g |
| Breast weight cooked | nm | | : nm | | 310 | g | 375 | g |
| Cooking time | 100 | min | 60 | min | 90 | min | 90 | min |

| | | | | |
|---|---|---|---|---|
| Cooked meat colour | off-white* | white* | pink/brown^{#} | white^{#} |
| Fat | fatty* | not fatty* | fatty^{#} | not fatty^{#} |
| Juiciness | dry* | juicy* | dry^{#} | juicy^{#} |
| Palatability preference | 0/7 | 7/7 | 0/4 | 4/4 |

nm not measured
*7/7;
^{#}4/4

These taste test results are consistent with the trends in the above sets of data, with the Zingibain-treated chickens having a lower cook loss (7%) and more cooked breast meat (18%) for the same carcass weight, and also having whiter meat and less intramuscular fat. The above taste testers, and other sets of taste testers for similar pairs of birds, unanimously determined that the Zingibain-treated chickens were more palatable, exhibiting less fat, more juice and whiter meat.

The consistent trends in the results across the measurements herein are of clear relevance for the chicken industry.

### EXAMPLE 10

### Trials of Zingibain in Horse Feed

The immune system of people and other animals in intensive training becomes sensitised, and consequently intolerant reactions to food are more common and severe. A high percentage of performance horses on feed do not digest their feed efficiently, leading to fermentation late in the intestine and the formation of acid. Faecal pH provides a measure of the acidity in the intestine attenuated by the faecal matter. Grazing horses normally have neutral faeces, but for performance horses on animal feed, the intestinal acidity increases and, if untreated, this can cause ulceration and other damage to the horse that impacts on the horse's general health and demeanour.

In this study using the ginger crush solution as a supplement to the feed, faecal pH was measured with a special electrode designed for measuring the pH of materials such as faeces to 0.02 pH, and 5 sets of pH values were recorded for each horse's daily faeces, usually with +/−0.1 pH reproducibility for a number of thoroughbred horses and one ‘warm-blood’ horse with a natural good digestion rate (Claude). The data are recorded in the following table.

Horses that are sensitive to animal feeds had faecal pHs about 6.4 without the supplement, but when the supplement was added to the feed, the pH increased to 7.0 or higher. If the ginger extract was not added, the pH fell on that day, and when the ginger extract was again added, the pH quickly increased to the neutral pH. If a ‘sensitive’ horse was spelled in a grassed paddock without access to animal feed, the faecal pH became neutral. Butazolidine was found to increase intestinal acidity.

TABLE 5
Daily pH Results

| Day | AOK | Mouse | Lutch | Claude¹ | Cooper |
|---|---|---|---|---|---|
| 1 | 7.3G | 6.9NG² | 7.2G | 6.7NG | — |
| 2 | 6.1NG | 6.4NG | 6.4NG | 7.0NG | 6.3NG |
| 3 | 5.5NG | 6.3NG | 6.1NG | 6.3NG | 6.5NG |
| 4 | 6.4G | 6.4NG | 6.5G | 6.8NG | 6.1G |
| 5 | 6.6G | 6.2NG | 7.1G | 6.9NG | 6.6G |
| 6 | 7.2G | 6.3NG | 6.2G³ | 6.6NG | 6.9G |
| 7 | 7.4G | 6.4NG | 6.2G | 6.6NG | 6.9G |
| 8 | 7.2G | 6.3NG | 6.2G | 6.6NG | 6.9G |
| 9 | 7.4G | 6.4NG | 6.2G | 6.6NG | 6.9G |
| 10 | 7.3G | 7.4P | 7.2G⁴ | 6.8NG | 7.8G |
| 11 | 7.4G | 7.5P | 6.7G⁴ | 6.8NG | 6.7G |
| 12 | 7.3G | — | 7.0G⁴ | 6.4NG | 7.3G |
| 13 | 6.8G | 7.5P | 7.1G⁴ | 7.0NG | 7.0G |
| 14 | 7.1G | 7.6P | 6.4G⁴ | 6.9NG | 7.3G |
| 15 | 7.2G | 6.9P | 6.7G⁴ | 7.0NG | 7.0G⁵ |
| 16 | 7.2G | 6.8P | 6.9G⁴ | 7.0NG | 6.9G |
| 17 | 6.9G | 6.8P | 6.8G⁴ | 6.8NG | 6.9G |

G: Ginger extract 150 mL added
NG: No ginger extract per day
P: In grassed paddock with no additional feed
1. ‘warm blood’ horse
2. had been on ginger extract prior to this date
3. injected with Butazolidine
4. double dose of ginger extract (300 mL)
5. Cooper was observed to eat bracken fern, and 4.00 Ketoprofen was given by intramuscular injection

It was concluded that the ginger extract is able to ensure the intestines of performance horses on animal feeds do not produce acid that can lead to ulceration and other disease conditions.

### EXAMPLE 11

### Degradation of blood clots by Zingibain

Fibrin and fibrinogen, from which fibrin is produced, have extremely important functions in animals. At the same time, however, these two proteins are associated with the occurrence of some of the more common diseases such as, for example, thrombosis.

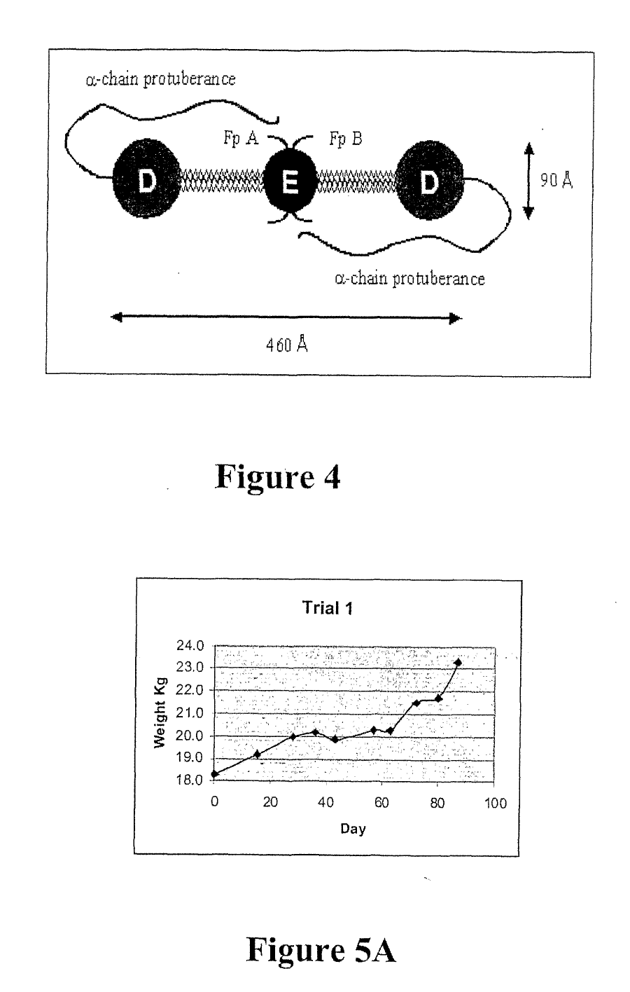

Fibrinogen is a plasma glycoprotein with a molecular weight of 340,000 Da. It is a dimeric protein, with each monomer unit being composed of disulfide linked chains Aα, Bβ and γ, forming a dimeric tri-nodular structure (refer to FIG. 4).

The α-helical coiled-coil domains of E₅, consisting of residues Aα50-78, Bβ85-114, and γ21-48, have an interesting structural feature. Coiled-coil sequences are usually characterized by a “heptad repeat”, where every third then fourth residue is usually a polar and closely packed in the core. In E₅, however, there is one three-residue deletion from the heptad repeat of each chain located at homologous positions (Aα65, Bβ100 and γ36) midway along the coiled-coil domain. These deletions or ‘stutters’ prevent the close packing. Furthermore, in this stutter region of the Bβ chains there are prolines at position 99 where the bend occurs with arginine preceding the prolines, facilitating hydrolysis by Zingibain.

The E₅ fragment is of relevance because it provides information about the topology of the fibrin clot. The endogenous hydrolysis of fibrinogen by thrombin at two Arg-Gly bonds liberates FpA from the two αN and FpB from the two Bβ chains. The liberation of the two FpA's results in the formation of two positively charged “knobs” on the E-domain consisting of Gly-Pro-Arg residues at positions 19-21 of the α-chains, which interact spontaneously with complementary “holes” pre-existing within the γ-chain C-termini on D-domains of neighbouring fibrin monomers (Hanna et al., V. J. Biochem., 23: 4681-4687; 1984). The liberation of FpB forms a GHR knob which is thought to contribute to the association between protofibrils (Laurent and Blomback, Acta Chem. Scand. 12: 1875-1877; 1958; Hantgan et al., “Fibrinogen structure and physiology” in “Hemostasis and Thrombosis: Basic Principles and Clinical Practice” Colman, R. W.; Hirsh, J.; Marder, V. J.; Salzman, E. W. (eds) J.B. Lippincott Company, Philadelphia, 1994, pp 277-300; Muller et al., J. Mol. Biol. 174: 369-384; 1984). Once the fibrin polymers are formed, they are covalently stabilized by transglutamination, a process catalyzed by coagulation factor XIIIa.

The γ-γ cross-linked fibrin molecules are degraded by plasmin through hydrolysis of Lys-X and Arg-X bonds located in the coiled-coil region with one Lys-Met bond hydrolyzed in the Aα-chain protuberances (Hantgan et al., 1994, supra). Depending on the degree of cross-linking, this produces monomeric D and E domains, dimeric D-domains (“D-dimers”), the Aα-chain protuberance, Bβ1-42, Bβ15-42 and lower molecular weight peptides from within the coiled-coil region (Hantgan et al., 1994, supra).

Fibrinogen and fibrin are rich in proline residues with hydrophilic residues adjacent. An SDS-PAGE electrophoresis study of the Zingibain degradation of purified human fibrinogen with added 2-mercaptoethanol to reduce the disulfide bridges showed that the individual α, β and γ-chains, which gave 3 bands in the region 60-52 KDa, were each totally degraded.

Furthermore, SDS-PAGE studies of Zingibain hydrolysis of human cross-linked fibrin with added 2-mercaptoethanol to break down the disulfide bridges (overnight incubation at room temperature) show the band from the γ-γ cross-linked chains completely removed. The dominant bands in the gel for the hydrolyzed fibrin are between 40 and 50 KDa. Polymerized cross-linked fibrin forms blood clots, thereby causing thrombosis. These results demonstrate that Zingibain is able to degrade blood clots efficiently.

Further, by hydrolyzing fibrinogen and possibly other members of the blood-clotting cascade, such as prothrombin, Zingibain is able to prevent the formation of blood clots, as shown by its ability to have significant effects on the prothrombin time assay for fresh citrated plasma, as set forth in Table 6.

TABLE 6
Normal citrated clotting times by prothrombin
time assay using tissue thromboplastin

| Incubation | 1 μg/mL Zingibain | 5 μg/mL Zingibain |
|---|---|---|
| Time (min) | Clotting Time (sec) | Clotting Time (Sec) |
| 0 | 14 | 14 |
| 15 | 16 | 18 |
| 30 | 32 | 46 |
| 60 | 48 | 84 |
| 120 | >200 | >200 |

These results indicate that, perhaps at levels as low as 1 ng/mL, Zingibain could limit the level of clots in blood.

### EXAMPLE 12

### Zingibain Reduces Inflammation

Fibrin and fibrinogen are closely associated with inflammation, which is defined broadly as a protective response of the body to tissue injury or destruction. Thrombin and factor XIIIa, which are generated immediately at the site of tissue damage, convert intra- and extravascular fibrinogen at the site to cross-linked fibrin. The fibrin meshwork entraps blood cells, limiting blood loss from the site and confines, to the site, inflammatory cells such as, for example, platelets, granulocytes, monocytes and lymphocytes, which would otherwise circulate.

Some of these cells as well as endothelial cells express on their outer surface cellular adhesion molecules (CAMs) that, when activated, have significant affinity for fibrin and fibrinogen. The platelet CAM, an integrin (α_{IIb}β₃), recognizes the final 12 residues of the γC-chains of fibrin and fibrinogen (Peerschke, Semin Hematol. 22: 241-259; 1985) and possibly the Arg-Gly-Asp sequences within fibrin and fibrinogen Aα-chains (Calvete, Proc. Soc. Exp. Biol Med. 208: 346-360, 1995). Neutrophils, monocytes and lymphocytes express at least two relevant CAM's, also integrins. One, α_{M}β₂, recognizes γ190-202 and γ377-395 within the fibrin and fibrinogen D-domains (Ugarova et al., J. Biol. Chem. 273: 22519-22527; 1998); the other, α_{χ}β₂, recognizes a Gly-Pro-Arg sequence at the fibrin αN-chains (Loike et al., Proc. Natl. Acad. Sci. USA 88: 1044-1048; 1991). Endothelial cells express two receptors for fibrin and fibrinogen: the integrin, αᵥβ₃, which recognizes Arg-Gly-Asp within the fibrin and fibrinogen Aα-chain (Hawiger, “Adhesive interactions of blood cells and the vascular wall” in “Hemostasis and Thrombosis: Principles and Clinical Practice” Colman, R. W.; Hirsh, J.; Marder, V. J.; Salzman, E. W. (eds) J. B. Lippincott Company, Philadelphia, 1994, pp 762-796), and intercellular adhesion molecule 1 (ICAM-1), a member of the immunoglobulin gene superfamily, recognizes γ117-133 (Languino et al., Proc. Natl. Acad. Sci. USA 92: 1505-1509; 1995).

Therefore, within the inflammation site, fibrin and fibrinogen are able to adhere to a variety of cells, keeping them in the location of the inflammation. In accordance with the present invention, this effect of fibrin and fibrinogen on inflammation may be abated by the degradation of fibrin and fibrinogen by Zingibain.

### EXAMPLE 13

### Zingibain Removes Skin Cancers

When Zingibain is applied topically in a cream base to human solar keratoses and to initial stages of basal cell carcinomas, the keratoses are destroyed leaving no scar and the scaly tissue from the incipient basal cell carcinomas is removed. The following five studies, and other similar experiments, provide evidence that Zingibain cream formulations may constitute a powerful, simple treatment of skin cancers.

(a) Patient: DG

The Zingibain cream formulation comprised ingredients: Aqua, glycerine 10%, cetearyl alcohol 10%, Z. officinale root extract (Zingibain) 0.3%, mineral oil, petrolatum, ceteareth 20.

All observations on history and treatment were provided by the registered nurse in care of the patient. Over a 7-year period, three skin lesions were removed by excision. No skin lesions were noted on arms or hands. Six years later, a skin lesion on the left-hand side nose (facing) was first observed, in January/February. It commenced with a scratch from a rose bush. Another lesion on the crest and right-hand side (facing) of the nose was not obvious prior to commencement of treatment on the left-hand lesion, which began six months later, in August.

This patient's history was as follows:

March: lesion larger, swab taken, and lesion after treatment with antibiotics resolved to a small spot.
May: lesion reappeared with large reddened area and with intermittent bleeding episodes.
June: Checked by the dermatologist in early June, who decided to reassess in late July regarding excision and grafting.
July: No further growth at this stage. The dermatologist left the lesion for further assessment and treatment in view of age of patient. Raised skin area on back of right hand and a reddened area on left forearm present by late July.

Zingibain cream was applied once daily by the registered nurse to upper aspect of nose, back of right hand and left forearm. Treatment commenced in August. The lesion on the left forearm was healed within three weeks, and the skin had attained normal tone and colour a month later. By the time the tube of cream became empty, approximately 10 weeks later, the lesion on the back of the right hand had healed with a small, hardened area still present. This hardened area had disappeared within two weeks, and the skin had assumed normal tone and colour. The lesion on the left-hand side (facing) of the nose had also healed completely by the time the tube of cream was empty, and the lesion on the crest and right-hand side (facing) only had a small area still raised.

In conclusion, a once-daily treatment of these skin cancers with Zingibain cream containing 0.3% Zingibain healed the lesions within a three month period. While a twice-daily application is usually recommended, the nurse was only able to provide the treatment once daily in this case. Nevertheless, the results were obvious.

(b) Patient: MS

The Zingibain paraffin cream formulation comprised ingredients: Aqua, glycerol 9%, light liquid paraffin 9%, soft white paraffin 4.5%, Z. officinale root extract (Zingibain) 0.3%, methyl hydroxybenzoate 0.2%, dichlorobenzyl alcohol 0.1%. The history of this patient was as follows:
October: Two solar keratoses on the side of the face were cryogenically removed by a dermatologist. However, following treatment, the wounds did not heal fully and continued to exhibit surface roughness.
December: The spots began to redden and increase in size. Zingibain paraffin cream was applied three times daily to the two spots.
February: When treatment commenced, the upper spot was approximately 3.5 mm in diameter and raised about 3 mM. The adjacent lower spot was approximately 4.5 mm in diameter and raised about 2 mm.
Upper spot: After one week, the upper spot began to contract and raise visibly, the surface becoming crusty and then flaking off. By the fourth week, the keratosis had disappeared, leaving healed skin.
Lower spot: The lower spot followed the same changes and disappeared 10 days later than the top spot, again leaving healed skin.

The dermatologist inspected the area of the spots the following March and October and found no sign of the keratoses.

(c) Patient: FH

Patient FH had a history of basal cell carcinomas on his arms and legs. These were periodically removed cryogenically.

Zingibain paraffin cream was applied twice a day to some incipient basal cell carcinomas for periods of four to six weeks. The Zingibain formulation comprised the same ingredients as listed in the previous case. The red, scaly, raised patches diminished in size over the period to leave clear skin with no scaliness and with no raised patches, and with either a much lighter red colour to the skin or no redness at all.

(d) Patient: DP

Patient DP had a recorded medical history of basal cell carcinomas, having had four excisions with the latest excision on the nose being unsuccessful and requiring extensive radiation therapy. Over the previous 10 years, it had been necessary to have liquid nitrogen treatment every six to 12 months for the removal of scaly skin patches and “sores” that are precursors of basal cell carcinomas.

Zingibain paraffin cream (as above) was applied twice a day for six weeks to an area on the right-hand side of the forehead that had extensive patches of scaly skin. After treatment, there were no signs of the scaliness, and no new scaly patches have appeared since (six months) in that area.

(e) Patient: FNB

Patient FNB has a very fair complexion and has had regular cryotherapy (liquid nitrogen treatment) to remove solar keratoses on his face for many years. In April 2003, the number of sun spots was so great that the dermatologist determined that it would be inappropriate to use cryotherapy, and changed the treatment to chemotherapy using a 5-Fluorouracil cream (Efudix) in conjunction with the synthetic corticosteroid, Advantan, to alleviate the severe discomfort and (temporary) disfiguration that are the usual side effects of Efudix therapy. Under this treatment (18 days), the keratoses became progressively much more inflamed red and scaly, and, after the 18 days therapy, the side-effects continued to increase in severity, despite the cortisone treatment.

To test Zingibain's anti-inflammatory and anti-cancer properties, FNB was invited to volunteer to try Zingibain treatment on one side of his face, and to continue to use the cortisone cream on the other side of the face to see if the Zingibain paraffin cream could more rapidly heal the lesions from the Efudix-treated keratoses, and in the longer term prevent the development of solar keratoses. The Zingibain cream was applied three times a day to the left-hand side of the face (cheek, nose, forehead and chin), and the cortisone cream to the right-hand side of the face commencing on 25 Apr. 2003 after the completion of the Efudix therapy.
Day 0: The left-hand cheek was photographed before treatment commenced.
Day 2: By the second day, the red, scaly keratoses on the left-hand side of the face had reduced in size and redness, with little sign of scaliness, whereas those lesions on the right-hand side of the face continued to worsen.
Day 5: This trend continued, and as a consequence on the fifth day, the person stopped using the cortisone cream on the right-hand side and applied the Zingibain cream on both sides of his face.
Day 9: The left-hand side of the face showed some pale pink patches on the skin, but no raised or scaly keratoses or any other side-effects from the Efudix treatment were apparent. The right-hand side also showed marked improvement.
Day 15: FNB noted that the keratoses had been effectively healed, and the side-effects of the Efudix therapy had been removed by the Zingibain treatment.
Days 16 & 17: FNB's face was inspected, and it was noted that almost all the keratoses had been healed leaving normal skin with no sign of lesion or redness. There were some pale pink spots remaining for a few of the lesions, and it was recommended that FNB continue to apply Zingibain cream until the face was totally clear (19 May 2003).

FNB has continued to use Zingibain cream on signs of potential solar keratoses on his arms and face with complete success.

### EXAMPLE 14

### Zingibain Aids Cell Harvesting

The extracellular domains of cell membrane proteins have a range of functions including acting as the receptor molecule for a signal to the cell or as the cell adhesion molecule (CAM) for various purposes. The expression of some of these molecules, or the mutation of these molecules, is associated with particular diseases such as cancers. Although fibrin is not an integral membrane protein, it binds to cell surfaces either through physico-chemical adsorption or through binding to specific CAMs. One of Zingibain's applications is to degrade the adsorbed fibrin, so that cultured cells do not adhere to their containers and can be harvested efficiently. Trypsin is commonly used for this but, because of its more general protease activity, in addition to cleaning off the fibrin from cell surfaces, it can also cleave off desirable cell membrane proteins that are required for the cell to function.

Zingibain's much greater protease specificity allows it to be used as a replacement for trypsin. It displays efficient removal of fibrin from the cell surfaces, but with less risk to cell membrane proteins unless those proteins have suitable proline residues exposed for hydrolysis.

Like other classical cadherins, E-cadherin is a single-pass, Type 1 cell surface glycoprotein which mediates cell-cell adhesion. E-cadherin is the principal cadherin found in epithelial tissues; in confluent epithelial cell monolayers, E-cadherin is found concentrated in adherens junctions as well as more diffusely throughout the lateral surfaces where cells adhere to one another.

For standard tissue culture procedures, where epithelial cells (e.g. MCF-7, MDCK) must be periodically passaged, treatment with a combination of trypsin and EDTA is commonly used to separate cells. This combination acts, at least in part, by cleaving E-cadherin and thus disrupting the cell-cell contacts. Of note, cleavage of E-cadherin by trypsin is sensitive to both trypsin activity and the extracellular calcium concentration. The cadherin ectodomain possesses calcium coordination sites and its conformation is calcium-sensitive. In the presence of calcium, the cadherin ectodomain adopts a rigid, rod-like orientation and is resistant to low concentrations of crystalline trypsin. If extracellular calcium is chelated, however, the ectodomain becomes sensitive (e.g. to 0.05% w/v crystalline trypsin).

For these experiments, it was desired to isolate individual epithelial cells, under conditions that preserve the cellular cadherin (this is quite independent of routine passaging of cells, where the cadherin is replenished). This can be quite difficult using the common cocktail of crystalline trypsin (0.05%) and calcium, at least partly because epithelial cells form calcium-independent desmosomes. Often cells are isolated only in sheets or clusters and vigorous trituration (with attendant shear damage) is necessary to separate them further. Accordingly, Zingibain was trialed as an alternative protease.

A range of Zingibain concentrations (0-5 mg/ml) was tested, diluted in Hanks balanced salt solution supplemented with 2 mM CaCl₂ (pH 7.4). MCF7 mammary epithelial cells (a well-differentiated breast cancer line which expresses endogenous E-cadherin) were grown to confluence and exposed to Zingibain for up to 10 min. By visual inspection, cells incubated in the higher concentrations (2-5 mg/ml) had separated by 5 min; at the lowest concentration good separation was seen by 10 min. Cells were collected by centrifugation and the total expression of E-cadherin assessed by Western blotting. No change in total E-cadherin levels was observed in any of the Zingibain-treated samples after 10 min incubation. The functional status of the cadherin was then assessed by the ability of cells to adhere and spread upon cadherin-coated substrata. For these studies, cells were isolated by exposure to 1 mg/ml Zingibain for 10 min. Adhesion and spreading of these isolated cells was excellent, suggesting that the functional competence of the cadherin was preserved.

### EXAMPLE 15

### Zingibain Inhibits a Virus

The cell-membrane proteins of viruses, such as neuraminidase and hemagglutinin of the influenza virus, are proline rich with multiple sites for hydrolysis by Zingibain. These proteins are essential for the infection process. Their cleavage inhibits the viral infection and proliferation.

An antiviral drug assay for testing the inhibitory activity of a drug against viruses was used for Zingibain with the mosquito-borne virus, Ross River Virus (RRV), at a dilution of 10⁻⁵ and 10⁻⁶. The virus was mixed with Zingibain at 0.020 mg/mL, and allowed to incubate at pH 7.2 for 2 hours. This was added to a confluent monolayer of Vero cells. The plaques produced by the virus were counted.

TABLE 7
Plaque assay of Ross River Virus incubated
with Zingibain (0.020 mg/mL) In Vero cells

| | Av. No. Plaques | Av. No. Plaques |
|---|---|---|
| RRV | with Zingibain | without Zingibain |
| −5 | 46 | 140 |
| −6 | 4 | 20 |

Higher concentrations of Zingibain could not be used in this type of assay, which relies on the cells being adhered to a glass surface, because Zingibain rounds up cells such as Vero cells from a surface. At 0.02 mg/mL, Zingibain inhibited RRV by up to 80%.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Abrol et al., Aust. J. Agric. Res. 22: 197-202, 1971
Calvete, Proc. Soc. Exp. Biol. Med. 208: 346-360, 1995
Choi et al., Biochem. 38: 11624-11633, 1999
Choi and Laursen, Eur. J. Biochem. 267: 1516-1526, 2000
Derbyshire et al., D. Phytochemistry 15: 3-24; 1976
Dionysius et al., J. Food Sci. 58: 780-784; 1993
Hanna et al., V. J. Biochem., 23: 4681-4687; 1984
Hantgan and Hermans, J. Biol. Chem. 254: 11272-11281; 1979
Hantgan et al., “Fibrinogen structure and physiology” in “Hemostasis and Thrombosis: Basic Principles and Clinical Practice” Colman, R. W.; Hirsh, J.; Marder, V. J.; Salzman, E. W. (eds) J.B. Lippincott Company, Philadelphia, 1994, pp 277-300
Hawiger, “Adhesive interactions of blood cells and the vascular wall” in “Hemostasis and Thrombosis: Principles and Clinical Practice” Colman, R. W.; Hirsh, J.; Marder, V. J.; Salzman, E. W. (eds) J.B. Lippincott Company, Philadelphia, 1994, pp 762-796
Hirsh et al. (eds) J.B. Lippincott Company, Philadelphia, 1994, pp 277-300
Ichikawa et al., J. Jpn. Soc. Food Nutr. 26: 377-383; 1973
Kirkman et al., J. Sci. Food Agric. 33: 115-127; 1982
Languino et al., Proc. Natl. Acad. Sci. USA 92: 1505-1509; 1995
Larkins, B. A. “Seed storage proteins: characterisation and biosynthesis” in “The Biochemistry of Plants” Stumpf, P. K.; Conn, E. E. (eds) Academic Press NY, Vol 6, pp 449-489
Laurent and Blomback, Acta Chem. Scand. 12: 1875-1877; 1958
Li et al., J. Animal Sci. 69: 4062; 1992
Loike et al., Proc. Natl. Acad. Sci. USA 88: 1044-1048; 1991
Muller et al., J. Mol. Biol. 174: 369-384; 1984
Ohtsuki et al., Biochim. Biophys. Acta 1243: 181-184; 1995
Partridge, “Considerations in the Formulation of Piglet Creep and Starter Feed” Technical Bulletin, American Soybean Association, 1997
Peerschke, Semin Hematol. 22: 241-259; 1985
Retzinger, “Fibrin(ogen) and Inflammation: Current Understanding and New Perspectives”2000. http://oz.uc.edu/˜retzings/fibrin( ).htm
Sheppy, “The current feed enzyme market and likely trends” in “Enzymes in Farm Animal Nutrition” Bedford, M. R.; Partridge, G. G. (eds) CABI Publishing Marlborough UK,
Spencer and Higgins, “Seed maturation and deposition of storage proteins” in “The Molecular Biology of Plant Development” Smith, H.; Grierson, D. (eds) Blackwell, Oxford
Stace, “Protein Content and Amino Acid Profiles of Honeybee-Collected Pollens” Bees‘N’Trees Consultants, Lismore, NSW, Australia, 1996, 2480
Thompson et al., J, Food Sci. 38: 652-655; 1973
Ugarova et al., J. Biol. Chem. 273: 22519-22527; 1998
Vader et al., Gastroenterology 122: 1729-1737; 2002

1-34. (canceled)

35. A method comprising;
administering to food an effective amount of Zingibain to affect food intolerance in a subject.

36. The method of claim 35, wherein said affect is selected from the group consisting of reducing food intolerance and removing food intolerance.

37. The method of claim 35, wherein said Zingibain is further effective to increase the palatability of food.

38. The method of claim 35, wherein said food is selected from the group consisting of a cereal, a legume, a bakery product, a pasta, a snack food, a nut containing food, an animal food product, and a dairy product containing food.

39. A method comprising;
utilizing an effective amount of Zingibain in the production of alcohol from cereal in order to increase the efficiency of said production of alcohol from said cereal.

40. A method for preparing a bakery product with cleaved gluten comprising;
a) mixing ingredients of said bakery product with an improver, said improver comprising Zingibain in an amount effective to cleave gluten,
b) mixing said bakery product and improver with further ingredients of said bakery product;
c) forming a dough or mixture or batter;
d) allowing the dough or mixture or batter to rest if required;
e) comminuting the dough if required; and
f) shaping and baking the dough or mixture or batter to form said bakery product with cleaved gluten.

41. The method of claim 40, wherein said bakery product is selected from the group consisting of breads, cakes, muffins, crumpets, English muffins, pizza bases, breakfast cereals, biscuits, cookies, doughnuts, scones, pancakes, pikelets and buns.

42. A food comprising;
an amount of Zingibain effective to increase the percentage water content from about 1% to 5%.

43. The food of claim 42, wherein said food is selected from the group consisting of pasta, a cereal containing food, and a snack food.

44. The food of claim 43, wherein said cereal containing food is a bakery product.

45. The food of claim 42, wherein said bakery product is bread comprising an improved texture or crust.

46. A food comprising;
an effective amount of Zingibain to affect food intolerance in a subject.

47. The food of claim 46, wherein the affect is selected from the group consisting of reducing food intolerance and removing food intolerance.

48. The food of claim 47, wherein said food is selected from the group consisting of a food component and an animal food product.

49. The food of claim 48, wherein said food component is selected from the group consisting of flour and whole grains.

50. The food of claim 46, wherein said food has reduced allergenicity to the relevant population in comparison to a corresponding untreated food.

51. The food of claim 46, wherein said food comprises an improvement selected from the group consisting of increased shelf-life, increased nutritional value and increased absorption in comparison to an untreated food.

52. A method comprising;
administering to an individual a therapeutically effective amount of Zingibain for the treatment of a condition selected from the group consisting of inflammatory disease and cancer.

53. The method of claim 52, wherein said inflammatory disease is selected from the group consisting of gluten intolerance, ulcerative colitis, inflammatory bowel disease, Crohn's disease, and infection by a pathogenic agent.

54. A method comprising;
administering an effective amount of Zingibain to a meat product to cleave prion proteins.

55. A method comprising;
utilizing an effective amount of Zingibain in cell harvesting to cleave fibrinogen.
